SEQUENCE LISTING (70 sequences)

SEQ ID NO: 1 - DNA, 80 bp, Artificial Sequence
  Description of Artificial Sequence Synthetic polynucleotide
tcgcacattccgcttctaccgggggggtcgagctgagtggatgcgaatctgtgggtgggccgtaagtccgtgtgtgcgaa

SEQ ID NO: 2 - DNA, 10 bp, Artificial Sequence
  Description of Artificial Sequence Synthetic Polynucleotide
ttcgcacaca

SEQ ID NO: 3 - DNA, 10 bp, Artificial Sequence
  Description of Artificial Sequence Synthetic Polynucleotide
tttttttttt

SEQ ID NO: 4 - DNA, 15 bp, Artificial Sequence
  Description of Artificial Sequence Synthetic Polynucleotide
ccccccccccaaaaa

SEQ ID NO: 5 - DNA, 10 bp, Artificial Sequence
  Description of Artificial Sequence Synthetic Polynucleotide
cccccccccc

SEQ ID NO: 6 - DNA, 10 bp, Artificial Sequence
  Description of Artificial Sequence Synthetic Polynucleotide
ccaacacaac

SEQ ID NO: 7 - DNA, 10 bp, Artificial Sequence
  Description of Artificial Sequence Synthetic Polynucleotide
ccaaccaacc

SEQ ID NO: 8 - DNA, 2 bp, Artificial Sequence
  Description of Artificial Sequence Synthetic Polynucleotide
cc

SEQ ID NO: 9 - DNA, 5 bp, Artificial Sequence
  Description of Artificial Sequence Synthetic Polynucleotide
aaaaa

SEQ ID NO: 10 - DNA, 9 bp, Artificial Sequence
  Description of Artificial Sequence Synthetic Polynucleotide
ggaaggaaa

SEQ ID NO: 11 - DNA, 9 bp, Artificial Sequence
  Description of Artificial Sequence Synthetic Polynucleotide
gagagagaa

SEQ ID NO: 12 - DNA, 10 bp, Artificial Sequence
  Description of Artificial Sequence Synthetic Polynucleotide
gagagagaaa

SEQ ID NO: 13 - DNA, 35 bp, Artificial Sequence
  Description of Artificial Sequence Synthetic Polynucleotide
ttttttttttccccccccccaaaaattcgcacaca

SEQ ID NO: 14 - DNA, 30 bp, Artificial Sequence
  Description of Artificial Sequence Synthetic Polynucleotide
ttttttttttccccccccccttcgcacaca

SEQ ID NO: 15 - DNA, 30 bp, Artificial Sequence
  Description of Artificial Sequence Synthetic Polynucleotide
ttttttttttccaacacaacttcgcacaca

SEQ ID NO: 16 - DNA, 30 bp, Artificial Sequence
  Description of Artificial Sequence Synthetic Polynucleotide
ttttttttttccaaccaaccttcgcacaca

SEQ ID NO: 17 - DNA, 22 bp, Artificial Sequence
  Description of Artificial Sequence Synthetic Polynucleotide
ttttttttttccttcgcacaca

SEQ ID NO: 18 - DNA, 25 bp, Artificial Sequence
  Description of Artificial Sequence Synthetic Polynucleotide
ttttttttttaaaaattcgcacaca

SEQ ID NO: 19 - DNA, 29 bp, Artificial Sequence
  Description of Artificial Sequence Synthetic Polynucleotide
ttttttttttggaaggaaattcgcacaca

SEQ ID NO: 20 - DNA, 29 bp, Artificial Sequence
  Description of Artificial Sequence Synthetic Polynucleotide
ttttttttttgagagagaattcgcacaca

SEQ ID NO: 21 - DNA, 30 bp, Artificial Sequence
  Description of Artificial Sequence Synthetic Polynucleotide
ttttttttttgagagagaaattcgcacaca

SEQ ID NO: 22 - DNA, 10 bp, Artificial Sequence
  Description of Artificial Sequence Synthetic Polynucleotide
cccccccggt

SEQ ID NO: 23 - DNA, 9 bp, Artificial Sequence
  Description of Artificial Sequence Synthetic Polynucleotide
aaaggaagg

SEQ ID NO: 24 - DNA, 9 bp, Artificial Sequence
  Description of Artificial Sequence Synthetic Polynucleotide
aagagagag

SEQ ID NO: 25 - DNA, 10 bp, Artificial Sequence
  Description of Artificial Sequence Synthetic Polynucleotide
aaagagagag

SEQ ID NO: 26 - DNA, 25 bp, Artificial Sequence
  Description of Artificial Sequence Synthetic Polynucleotide
cccccccggtaaaaatttttttttt

SEQ ID NO: 27 - DNA, 20 bp, Artificial Sequence
  Description of Artificial Sequence Synthetic Polynucleotide
cccccccggttttttttttt

SEQ ID NO: 28 - DNA, 30 bp, Artificial Sequence
  Description of Artificial Sequence Synthetic Polynucleotide
cccccccggtccaaccaacctttttttttt

SEQ ID NO: 29 - DNA, 30 bp, Artificial Sequence
  Description of Artificial Sequence Synthetic Polynucleotide
cccccccggtcccccccccctttttttttt

SEQ ID NO: 30 - DNA, 29 bp, Artificial Sequence
  Description of Artificial Sequence Synthetic Polynucleotide
cccccccggtaaaggaaggtttttttttt

SEQ ID NO: 31 - DNA, 29 bp, Artificial Sequence
  Description of Artificial Sequence Synthetic Polynucleotide
cccccccggtaagagagagtttttttttt

SEQ ID NO: 32 - DNA, 30 bp, Artificial Sequence
  Description of Artificial Sequence Synthetic Polynucleotide
cccccccggtaaagagagagtttttttttt

SEQ ID NO: 33 - DNA, 31 bp, Artificial Sequence
  Description of Artificial Sequence Synthetic Polynucleotide
cacccggtagaaaagagagagtttttttttt

SEQ ID NO: 34 - DNA, 29 bp, Artificial Sequence
  Description of Artificial Sequence Synthetic Polynucleotide
cccggtagaaaagagagagtttttttttt

SEQ ID NO: 35 - DNA, 28 bp, Artificial Sequence
  Description of Artificial Sequence Synthetic Polynucleotide
ccggtagaaaagagagagtttttttttt

SEQ ID NO: 36 - DNA, 31 bp, Artificial Sequence
  Description of Artificial Sequence Synthetic Polynucleotide
cacacggtagaaaagagagagtttttttttt

SEQ ID NO: 37 - DNA, 28 bp, Artificial Sequence
  Description of Artificial Sequence Synthetic Polynucleotide
ccccccggtaagagagagtttttttttt

SEQ ID NO: 38 - DNA, 27 bp, Artificial Sequence
  Description of Artificial Sequence Synthetic Polynucleotide
cccccggtaagagagagtttttttttt

SEQ ID NO: 39 - DNA, 26 bp, Artificial Sequence
  Description of Artificial Sequence Synthetic Polynucleotide
ccccggtaagagagagtttttttttt

SEQ ID NO: 40 - DNA, 12 bp, Artificial Sequence
  Description of Artificial Sequence Synthetic Polynucleotide
cacccggtagaa

SEQ ID NO: 41 - DNA, 10 bp, Artificial Sequence
  Description of Artificial Sequence Synthetic Polynucleotide
cccggtagaa

SEQ ID NO: 42 - DNA, 9 bp, Artificial Sequence
  Description of Artificial Sequence Synthetic Polynucleotide
ccggtagaa

SEQ ID NO: 43 - DNA, 12 bp, Artificial Sequence
  Description of Artificial Sequence Synthetic Polynucleotide
cacacggtagaa

SEQ ID NO: 44 - DNA, 9 bp, Artificial Sequence
  Description of Artificial Sequence Synthetic Polynucleotide
ccccccggt

SEQ ID NO: 45 - DNA, 8 bp, Artificial Sequence
  Description of Artificial Sequence Synthetic Polynucleotide
cccccggt

SEQ ID NO: 46 - DNA, 7 bp, Artificial Sequence
  Description of Artificial Sequence Synthetic Polynucleotide
ccccggt

SEQ ID NO: 47 - DNA, 25 bp, Artificial Sequence
  Description of Artificial Sequence Synthetic Polynucleotide
ttttttttttaaaaacccccccggt

SEQ ID NO: 48 - DNA, 20 bp, Artificial Sequence
  Description of Artificial Sequence Synthetic Polynucleotide
ttttttttttcccccccggt

SEQ ID NO: 49 - DNA, 30 bp, Artificial Sequence
  Description of Artificial Sequence Synthetic Polynucleotide
ttttttttttccaaccaacccccccccggt

SEQ ID NO: 50 - DNA, 30 bp, Artificial Sequence
  Description of Artificial Sequence Synthetic Polynucleotide
ttttttttttcccccccccccccccccggt

SEQ ID NO: 51 - DNA, 29 bp, Artificial Sequence
  Description of Artificial Sequence Synthetic Polynucleotide
ttttttttttggaaggaaacccccccggt

SEQ ID NO: 52 - DNA, 29 bp, Artificial Sequence
  Description of Artificial Sequence Synthetic Polynucleotide
ttttttttttgagagagaacccccccggt

SEQ ID NO: 53 - DNA, 30 bp, Artificial Sequence
  Description of Artificial Sequence Synthetic Polynucleotide
ttttttttttgagagagaaacccccccggt

SEQ ID NO: 54 - DNA, 76 bp, Artificial Sequence
  Description of Artificial Sequence Synthetic Polynucleotide
tagggaagagaaggacatatgatcgtaccgcaagtgacgtgtccgtgccgtgattgactagtacatgaccacttga

SEQ ID NO: 55 - DNA, 12 bp, Artificial Sequence
  Description of Artificial Sequence Synthetic Polynucleotide
tcaagtggtcat

SEQ ID NO: 56 - DNA, 4 bp, Artificial Sequence
  Description of Artificial Sequence Synthetic Polynucleotide
caaa

SEQ ID NO: 57 - DNA, 6 bp, Artificial Sequence
  Description of Artificial Sequence Synthetic Polynucleotide
caaaac

SEQ ID NO: 58 - DNA, 37 bp, Artificial Sequence
  Description of Artificial Sequence Synthetic Polynucleotide
ttttttttttccccccccccaaaaatcaagtggtcat

SEQ ID NO: 59 - DNA, 32 bp, Artificial Sequence
  Description of Artificial Sequence Synthetic Polynucleotide
ttttttttttcccccccccctcaagtggtcat

SEQ ID NO: 60 - DNA, 27 bp, Artificial Sequence
  Description of Artificial Sequence Synthetic Polynucleotide
tttttttttttcaaatcaagtggtcat

SEQ ID NO: 61 - DNA, 29 bp, Artificial Sequence
  Description of Artificial Sequence Synthetic Polynucleotide
tttttttttttcaaaactcaagtggtcat

SEQ ID NO: 62 - DNA, 24 bp, Artificial Sequence
  Description of Artificial Sequence Synthetic Polynucleotide
ttttttttttcctcaagtggtcat

SEQ ID NO: 63 - DNA, 27 bp, Artificial Sequence
  Description of Artificial Sequence Synthetic Polynucleotide
ttttttttttaaaaatcaagtggtcat

SEQ ID NO: 64 - DNA, 23 bp, Artificial Sequence
  Description of Artificial Sequence Synthetic Polynucleotide
gaaaagtgctctgtgaactctat

SEQ ID NO: 65 - DNA, 33 bp, Artificial Sequence
  Description of Artificial Sequence Synthetic Polynucleotide
ttttttttttgaaaagtgctctgtgaactctat

SEQ ID NO: 66 - DNA, 15 bp, Artificial Sequence
  Description of Artificial Sequence Synthetic Polynucleotide
ttttttttttaaaaa

SEQ ID NO: 67 - DNA, 15 bp, Artificial Sequence
  Description of Artificial Sequence Synthetic Polynucleotide
ttttttttttaaaaa

SEQ ID NO: 68 - DNA, 33 bp, Artificial Sequence; identical to SEQ ID NO: 65
  Description of Artificial Sequence Synthetic Polynucleotide

SEQ ID NO: 69 - DNA, 13 bp, Artificial Sequence
  Description of Artificial Sequence Synthetic Polynucleotide
ttcgcacacacgg

SEQ ID NO: 70 - DNA, 32 bp, Artificial Sequence
  Description of Artificial Sequence Synthetic polynucleotide
ttttttttttgagagagaattcgcacacacgg

(19) United States
(12) Patent Application Publication
(10) Pub. No.: US 2022/0298570 A1
(43) Pub. Date: Sep. 22, 2022

(54) OPTIMIZED NUCLEIC ACID PROBES FOR ANALYTE DETECTION

(71) Applicant: DOTS Technology Corp., Natick, MA (US)

(72) Inventors: Adi Gilboa-Geffen, Wayland, MA (US); Valerie Villareal, Boston, MA (US); Patrick Murphy, Allston, MA (US); Nhat Nam Trinh, Quincy, MA (US)

(21) Appl. No.: 17/636,403

(22) PCT Filed: Aug. 20, 2020
(86) PCT No.: PCT/US20/47093

§ 371 Date: Feb. 18, 2022

Related U.S. Application Data
(60) Provisional application No. 62/889,081, filed on Aug. 20, 2019.

(51) Int. Cl. C12Q 1/6876; C12Q 1/6834

(52) U.S. Cl. CPC C12Q 1/6876; C12Q 1/6834; C12Q 2600/166

(57) ABSTRACT

The present disclosure relates to optimized nucleic acid probes, nucleic acid chips, and assays and methods for detection of an analyte of interest in a sample, for example, an allergen in a food sample.

### CROSS REFERENCE TO RELATED APPLICATIONS

The present disclosure claims priority of U.S. Provisional Application Ser. No. 62/889,081, filed Aug. 20, 2019, the contents of which are incorporated herein by reference in their entirety.

### REFERENCE TO THE SEQUENCE LISTING

The present application is being filed along with a Sequence Listing in electronic format. The Sequence Listing is provided as a file entitled 20661012PCTSEQLST.txt, created on Aug. 20, 2020 which is 16,734 bytes in size. The information in the electronic format of the sequence listing is incorporated herein by reference in its entirety.

### FIELD OF THE DISCLOSURE

The present disclosure relates to nucleic acid probes, solid substrates with nucleic acid probes immobilized thereto (e.g., DNA chips) and processes and methods of use thereof.

### BACKGROUND OF THE DISCLOSURE

Biosensors comprising Nucleic acid (e.g., short oligonucleotides) coated chips, e.g., DNA chips, have come into widespread use for detection of an analyte (e.g., a cell, a bacteria, a virus, a nucleic acid molecule, a protein, a toxin, a peptide, a lipid, and a sugar) in a sample, and analyzing sequences and gene mapping in the field of genomics and medical diagnosis. The hybridization mechanism as a representative example, is a method for capturing a target nucleic acid molecule with a nucleic acid probe by utilizing the interaction of complementary nucleic acid strands (hybridization), and determining directly or indirectly the presence of a target analyte. A single stranded nucleic acid molecule having a sequence of all or part of the target nucleic acid molecule is commonly used as the probe, and the sensor chip for detection of the target nucleic acid molecule is formed by immobilizing (e.g., by a covalent bond, ionic bond, adsorption, or biological specific binding) the probe on a solid phase substrate (e.g., a glass chip and a plastic).

The process of making a DNA chip comprising a solid substrate with a nucleic acid probe immobilized thereto, often requires that nucleic acid probes and/or the surface to which the nucleic acid probes are immobilized are chemically modified to facilitate the attachment. Nucleic acid and/or surface modifications can affect the efficiency of probe attachment, density of immobilized probes on the surface, cross-interaction between probe sequences, structural state of the probes and hybridization with target nucleic acid sequences, and spotting pattern, etc.

For example, surface oligonucleotide density could affect a wide variety of applications of DNA chips, like thermodynamic stability of double-stranded nucleic acid molecules formed during a detection assay. The higher the density of nucleic acid probes immobilized on the solid substrate, the more the amount of target nucleic acid molecules that can be captured by hybridization to the complementary nucleic acid probes. Nonetheless, there is the concern that the high density of nucleic acid probes may inhibit hybridization. Therefore, it is necessary to adjust the density of the nucleic acid probes on the surface of the solid substrate to the optimal level in order to efficiently capture target nucleic acid molecules at maximum amount. In some cases, each spot pitch and the spots pattern on the surface of a chip can influence the hybridization efficiency.

Nucleic acid probes of difference nucleotide lengths and compositions can affect the chip features. The present disclosure provides optimized nucleic acid probes, which simplify the immobilization process using UV directly cross-linking, and to ensure high density and uniformed distribution of the probes on a solid substrate.

### BRIEF DESCRIPTION OF THE DRAWINGS

FIG. 1 shows representative formula of the nucleic acid probe comprising a linker (A) and uniquely specific nucleic acid probe sequence (C), and optionally a spacer (B) between the linker (A) and the probe sequence (C).

FIGS. 2A to 2D demonstrate 2D structures of a target nucleic acid sequence, i.e., a signaling polynucleotide that binds to a peanut allergen (AraHl SPN; SEQ ID NO. 1).

FIGS. 3A and 3B demonstrate exemplary patterns of nucleic acid probes immobilized onto the surface of a solid substrate, e.g., a chipannel.

FIG. 4 is representative images of UV-spotted chipannel and expoysaline-coated chipannel after incubating with food samples and washing.

### SUMMARY OF THE DISCLOSURE

The present disclosure relates to optimized nucleic acid probes suitable for immobilization on a carrier such as a solid substrate, and for analysis of an analyte of interest in a sample. In one aspect, this present disclosure provides a nucleic acid probe including a uniquely specific oligonucleotide probe sequence (i.e., probe sequence), a linker sequence and optionally a spacer sequence, and a solid substrate with at least one nucleic acid probe immobilized thereto (e.g., a DNA chip). In another aspect, the present disclosure provides methods for production and use of such nucleic acid probes and DNA chips. The disclosed probes are optimized to improve the immobilization of the probes to a solid substrate, to reduce or eliminate self-assembly of the probes, and to reduce background signals.

In some embodiments, the present nucleic acid probes are produced by a method that includes joining a uniquely specific oligonucleotide probe sequence, a spacer sequence and a linker sequence in a pre-determined order.

In some embodiments, the present nucleic acid probe comprises a uniquely specific oligonucleotide probe sequence that is complementary to the sequence or a portion of the sequence of a target nucleic acid sequence. In some examples, the target nucleic acid sequence specifically binds to an analyte of interest such as bacteria, fungi, tissue, cell, protein, nucleic acid molecule, lipid, sugar, toxin and chemical compound. In one preferred embodiment, the analyte of interest is a protein, e.g., an allergen like a food allergen. The target nucleic acid sequence may be an aptamer that specifically binds to an analyte of interest (e.g., allergen). The aptamer may be further modified to increase its specificity and affinity to the analyte of interest. A target nucleic acid sequence (e.g., aptamer) may be used as a detection agent to detect a target analyte in a sample.

The uniquely specific oligonucleotide probe sequence is about 80%, 85%, 90%, 91%, 92%, 93%, 94%, 95%, 96%, 97%, 98%, 99%, or 100% complementary to the sequence, a portion of the sequence of the target nucleic acid sequence, e.g., the sequence of an aptamer against an allergen.

The uniquely specific oligonucleotide probe sequence may include 5-30 nucleotides, or 5-15 nucleotides, or 10-20 nucleotides. The uniquely specific oligonucleotide probe sequence may include about 5, 6, 7, 8, 9, 10, 11, 12, 13, 14, 15, 16, 17, 18, 19, 20, 21, 22, 23, 24, 25, 26, 27, 28, 29, or 30 nucleotides.

In some embodiments, the linker sequence is attached to one end of the uniquely specific oligonucleotide probe sequence, either the 3′ end or 5′end of the probe sequence. In some examples, the linker sequence comprises about 5-20 nucleotides, for example, 5, 6, 7, 8, 9, 10, 11, 12, 13, 14, 15, 16, 17, 18, 19 or 20 nucleotides. In some examples, the linker sequence comprises a poly (T)n sequence wherein then is from 5 to 15. As a non-limiting example, the linker sequence may comprise a poly(T)(10) (5′TTTTTTTTTT3′; SEQ ID NO. 3).

In some embodiments, the present nucleic acid probe can optionally comprise a spacer sequence. The spacer sequence may be inserted between the linker sequence and the uniquely specific oligonucleotide probe sequence. The spacer sequence may spatially separate the linker sequence and the probe sequence and maintain the structural state of the probe for hybridization between the probe and its target nucleic acid sequence. As a non-limiting example, the spacer sequence may be selected from the group consisting of SEQ ID Nos. 11 and 23.

In another aspect, the present disclosure relates to a sensor chip that comprises a solid substrate (e.g., a glass chip, a plastic chip, etc.) with at least one nucleic acid probe immobilized thereto. Complementary target nucleic acids are specifically recognized through hybridization with the nucleic acid probes on the substrate. The probes may be immobilized to the substrate by UV light cross-linking. In some embodiments, short control oligonucleotide probes are spotted onto the solid substrate as well. The control sequences are designed for measuring a total protein as internal control of a detection assay. The nucleic acid probes and control oligonucleotide sequences are spotted on the chip surface in a specific pattern. In one preferred embodiment, a chip comprises a plurality of spots with nucleic acid probes and a plurality of spots with control oligonucleotide sequences. In some examples, the solid substrate may contain more than two subdivided probe sets. A first and second probe sets comprise a plurality of nucleic acid probes exhibiting complementarity with a target nucleic acid sequence (e.g., a SPN).

In one preferred embodiment, the sensor chip is a chipannel comprising a specialized sensor area where the nucleic acid probes and control sequences are immobilized thereto. The nucleic acid probes and control sequences form a reaction panel and a control panel on the chipannel, respectively.

In another aspect of the present disclosure, a detection kit is provided comprising nucleic acid probes, chipannels, reagents (e.g., hybridization and wash buffers) and instructions.

In further another aspect of the present disclosure, methods of using the disclosed nucleic acid probes and chips including detection, in some examples, and quantification of an analyte of interest in a sample such as an allergen, are provided. The method comprises (a) providing a complex formed from (i) a sample suspected of containing the analyte of interest and (ii) a nucleic acid based detection agent in a condition allowing the binding of the analyte to the detection agent, wherein the detection agent comprises a nucleic acid sequence that binds to the analyte of interest; (b) contacting the complex of the analyte of interest and the detection agent to a nucleic acid probe immobilized to a solid substrate, wherein the probe comprises an oligonucleotide probe sequence that is complementary to the sequence or a portion of the sequence of the detection agent; (c) applying a detection module to the solid substrate for detecting a signal from the detection agent and the oligonucleotide probe, wherein if the analyte is not present in the sample, the detection agent not bound to the analyte is coupled to the solid substrate via the direct hybridization between the probe sequence and the target sequence of the detection agent; and (d) measuring the amount of the detection agent wherein the amount of the detection agent indicates where or not the analyte of interest is present in the sample.

In some embodiments, the detection method comprises contacting the probes with a mixture of a sample suspected of including the analyte of interest and a target nucleic acid agent (e.g., aptamer) that binds to the analyte of interest under conditions sufficient to permit hybridization between the probes and the target nucleic acid agent (e.g., aptamer). Resulting hybridization is detected, wherein the presence of hybridization indicates the presence or quantification of the analyte of interest in the sample.

### DETAILED DESCRIPTION OF THE DISCLOSURE

The foregoing has outlined rather broadly the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure. Unless defined otherwise, all technical and scientific terms used herein have the same meaning as commonly understood by one of ordinary skill in the art to which this disclosure belongs. In the case of conflict, the present description will control.

### Definitions

To more clearly and concisely describe the subject matter of the claimed disclosure, the following definitions are provided for specific terms, which are used in the following description and the appended claims. Throughout the specification, exemplification of specific terms should be considered as non-limiting examples.

As used herein, the terms “nucleic acid,” “oligonucleotide” and “polynucleotide” are used interchangeably. A nucleic acid molecule is a polymer of nucleotides consisting of at least two nucleotides covalently linked together. A nucleic acid molecule is a DNA (deoxyribonucleotide), a RNA (ribonucleotide), as well as a recombinant RNA and DNA molecule or an analogue of DNA or RNA generated using nucleotide analogues. The nucleic acids may be single stranded or double stranded, linear or circular. The term also comprises fragments of nucleic acids, such as naturally occurring RNA or DNA which may be recovered using the extraction methods disclosed, or artificial DNA or RNA molecules that are artificially synthesized in vitro. Molecular weights of nucleic acids are also not limited, may be optional in a range from several base pairs (bp) to several hundred base pairs, for example from about 2 nucleotides to about 1,000 nucleotides. As non-limiting examples, a nucleic acid molecule may comprise 2, 3, 4, 5, 6, 7, 8, 9, 10, 12, 15, 20, 25, 30, 35, 40, 45, 50, 55, 60, 65, 70, 75, 80, 85, 90, 95, 100, 110, 120, 130, 140, 150, 200, 250, 300, 350, 400, 450, 500, 550, 600, 650, 700, 750, 800, 850, 900, or 1,000 nucleotides. The nucleic acid may be chemically modified. The term “modified”, “modifying” or “modification” when used herein in reference to a nucleic acid molecule means one or more nucleotides are modified, e.g., structural modifications of nucleotides which do not occur naturally, replacement by a nucleoside analogue, and chemical modification of the sugar-phosphate backbone, etc. In some examples, modified nucleic acid molecules may comprise non-standard or modified nucleosides.

As used herein, the term “probe” refers to proteins (including peptides), nucleic acids, sugar chains (including glycoconjugates), lipids (including conjugated lipids), and the like biopolymers. Specifically, the probe includes enzymes, hormones, pheromones, antibodies, antigens, haptens, peptides, synthetic peptides, DNA, synthetic DNA, RNA, synthetic RNA, DNA/RNA hybrids, PNA, synthetic PNA, gangliosides, oligonucleotides, aptamers, lectins, etc. In the context of the present disclosure, the probe is a nucleic acid probe comprising a nucleic acid sequence that specifically is complementary to a target nucleic acid sequence.

As used herein, the term “target nucleic acid” refers to a nucleic acid (such as DNA or RNA) sequence of either natural or synthetic origin that is desired to bind to an analyte of interest that is to be analyzed and the target nucleic acid is to be captured by the nucleic acid probe. In the context of the present disclosure, a target nucleic acid sequence may be an aptamer that is selected by standard SELEX methods, or a signaling polynucleotide (SPN) derived from the aptamer.

As used herein, the term “complementary” generally refers to specific nucleotide duplexing to form canonical Watson-Crick base pairs, as is understood by those skilled in the art. For example, two nucleic acid strands or parts of two nucleic acid strands are said to be complementary or to have complementary sequences in the event that they can form a perfect base-paired double helix with each other. The term “hybridization” refers to non-covalent bonding through base pairings between A and T, and G and C.

As used herein, the term “linker” means a molecule or moiety that is attached to one end of a nucleic acid probe sequence. The linker exists between the nucleic acid probe sequence and the solid substrate, links the probe to the substrate and provides spacing between the two moieties such that they are able to function in their intended manner. The moiety can be a chemical compound, a peptide, or a short oligonucleotide sequence. In the context of the present invention, the linker is a short oligonucleotide sequence that is attached to either the 5′ end or 3′ end of an oligonucleotide probe sequence.

As used herein, the term “spacer” refers to a molecule or moiety that increases the space between two molecules or moieties. Spacer of different sizes and lengths may be inserted into a nucleic acid probe sequence and the linker sequence. The spacer may spatially separate the probe sequence and the substrate and maintain the structural state of the nucleic acid probe, for example decreasing the tendency of forming intramolecular self-dimer and hairpins. In the context of the present invention, the spacer is a short oligonucleotide sequence between the linker and the oligonucleotide probe.

As used herein, the term “solid substrate” is not particularly limited as long as the substrate does not prevent the immobilization of nucleic acid molecules, and any kind of solid phase substrate may be used. The material, the number of layers, and the types and thicknesses of the solid substrate may depend on the immobilization methods used and on the signal detection means adopted in order to detect the target nucleic acid molecule. Exemplary substrates may include but are not limited to glass substrate, metal substrate (e.g., gold, silver, copper, aluminum, platinum, aluminum oxide, SrTiO₃, LaAl₃, NdGaO₃, and ZrO₂), silicon substrate (e.g., silica oxide) and polymer resin substrate (e.g., polyethylene terephthalate, polycarbonate, polystyrene, cyclic olefin copolymer (COC), cyclic olefin polymer (COP), polypropylene), etc. The solid substrate may be a substrate comprising a single material of those listed above, or may comprise a thin film on the surface of the substrate consisting of at least one material other than the material of the substrate. In some examples, the substrate may be a porous substrate (e.g., a nylon). In other examples, the substrate may be a non-porous substrate.

The solid substrate as mentioned above may be introduced functionalized groups that can improve the attachment of nucleic acid probes. The functionalized groups may include but are not limited to, amine-groups and carboxyl groups.

As used herein, the term “chip” could be understood to be any three-dimensional shape. The substrate may be any types of materials that are suitable for nucleic acid immobilization as discussed above. The materials used as a chip substrate may have the desirable characteristics including optical characteristics, e.g., flatness, transparency, a well-defined optical absorption spectrum, minimal auto-fluorescence, high reflectivity; and chemical characteristics, e.g., surface reactivity that permits covalent linkages. Non-limiting examples of suitable substrates include inorganic materials, e.g., silicon, glasses and ceramic (such as low-temperature cofired ceramic (LTCC)), polymer substrates, composites and paper (Nge et al., 2013 and Wu et al., 2013). Polymers may include elastomers, e.g., polydimethylsiloxane (PDMS; dimethicone), polyester (e.g., thermoset polyester (TPE)) ; thermoplastic polymers, e.g., polystyrene (PS),polycarbonate (PC), poly-methyl methacrylate (PMMA), and poly-ethylene glycol diacrylate (PEGDA), perfluorinated compounds/polymers (such as perfluoroalkoxy (Teflon PFA), fluorinated ethylenepropylene (Teflon FEP), and polyfluoropolyether diol methacrylate (PFPE-DMA)), and polyurethane (PU); and thermosets, and polyimide and acrylic. paper, a flexible cellulose-based material, composite materials, e.g., amorphous material, cyclic olefin polymers (COP), polymers based on cyclic olefin monomers and ethene, such as cyclic olefin copolymer (COC). In some examples, the chips are plastic chips: which have excellent microfabrication properties and are more easily amenable to integration into low-cost, portable analysis systems.

As used herein, the terms “DNA chip,” “oligonucleotide chip” and “nucleic acid chip” are used interchangeably. A nucleic acid chip means a probe immobilized carrier such as a solid substrate with arrays of nucleic acid probes that are tethered to the surfaces of substrates for capture of targets, e.g., complementary analyte DNAs and proteins. A nucleic acid chip may be created by producing surface-immobilized probes via direct, on-chip synthesis of nucleic acids, or by attaching pre-synthesized oligonucleotides that are chemically modified to effect surface immobilization. In some embodiments, the pre-synthesized nucleic acid probes are linked to the solid phase substrate via generating covalent bonds. Therefore, the nucleic acid probes are tightly immobilized on the surface, providing high stability of the arrays and reproducibility of the data obtained. In some cases, both nucleic acid probes and solid surfaces are usually modified with reactive functional groups to allow chemical reactions to form covalent bonds between the probe and surface. Commonly used functional groups include but are not limited to carboxyl, phosphate, aldehyde and amino groups. For example, amino groups, can be employed for both the probe and the surface because of its easy preparation, stable functionality and wide applicability. The solid surface may be modified with amino groups to generate a NH₂-functionalized surface, subsequently subjected to chemical activation by use of homo-bifunctional linkers such as disuccinimidyl glutarate (DSG), phenylene diisothiocyanate (PDC). In other examples, the probe DNA oligonucleotides with carboxyl or phosphate groups at the ends are immobilized on the NH₂-functionalized surface, dehydration reagents such as dicyclohexylcarbodiimide (DCC), 1-ethyl-3-(3-dimethylaminopropyl)carbodiimide (EDC), etc. are employed usefully for their activation. In other examples, the pre-synthesized oligonucleotides may be immobilized to an untreated surface by UV light irradiation.

As used herein, the term “sample” include any sources suspected to contain an analyte of interest. A sample may include a food sample and a biological sample. The term “biological sample” refers to samples originating from or obtained from biological material. Biological materials include living species of eubacteria, eukaryotes and archaea as well as viruses.

As used herein, the term “analyte” refers to any element, component or compound which may be present in a sample and the presence and/or the concentration of which may be of interest for a user. An analyte can be a bacterium, a virus, a cell, a nucleic acid, a protein, a sugar, a lipid and a chemical compound. Particularly the analyte may be an allergen in a sample, e.g., a food allergen in a food sample. Common food allergens may include but are not limited to peanut, tree nuts, milk, egg white, wheat, soy, fish and sea food. In other examples, the analyte may be an element, component or compound which may be present in a biological sample, including but not limited to nucleic acids (e.g., DNA, mRNA, tRNA, siRNA), proteins (e.g., antibodies), lipids and sugars.

### Compositions

The present disclosure provides optimized nucleic acid probes, nucleic acid chips, agents for capturing target nucleic acid sequences and methods of use thereof for detection of an analyte of interest in a sample. The nucleic acid probes are optimized for immobilizing to a carrier such as a solid substrate and for capturing a target nucleic acid sequence. Particularly the probes immobilized on a chip can capture its target nucleic acid molecule in a state that the target sequence is in contact with an analyte. The probe comprises a uniquely specific oligonucleotide probe sequence that hybridizes to a target sequence that binds to an analyte of interest. The analyte can be an allergen such as a food allergen in a food sample.

In some embodiments, the nucleic acid probe may include suitable chemical modifications that would allow the probe to be bound to a solid substrate. Suitable, but non-limiting modifications include functional groups such as thiols, amines, carboxylic acids, maleimide, and dienes. Other methods such as hapten interactions may be used. The probes can be prepared by any suitable means, including chemical synthesis and chemical synthesis on solid substrates. In some embodiments, the nucleic acid probe is specifically modified for direct attachment to different types of plastics without any chemical modification of the surface. As a non-limiting example, the probe is printed on a solid substrate through simple UV light cross-linkage.

### 1. Nucleic Acid Probes

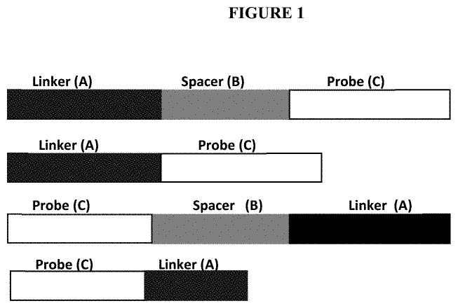

The present disclosure provides optimized nucleic acid probes that are suitable for printing on a solid substrate directly. The probe is optimized for immobilization to a solid substrate and for binding to a target nucleic acid sequence. In some embodiments, the probe comprises a short single stranded oligonucleotide probe sequence (e.g., probe C shown in FIG. 1) comprising 5-50 nucleotides, or 5-30 nucleotides, or 5-25 nucleotides, or 10-25 nucleotides, or 8-15 nucleotides, or 10-20 nucleotides. As non-limiting examples, the oligonucleotide probe sequence may comprise at least 5 nucleotides, or at least 6 nucleotides, or at least 7 nucleotides, or at least 8 nucleotides, or at least 9 nucleotides, or at least 10 nucleotides, or at least 11 nucleotides, or at least 12 nucleotides, or at least 13 nucleotides, or at least 14 nucleotides, or at least 15 nucleotides, at least 16 nucleotides, or at least 17 nucleotides, or at least 18 nucleotides, or at least 9 nucleotides, or at least 20 nucleotides, or at least 21 nucleotides, or at least 22 nucleotides, or at least 23 nucleotides, or at least 24 nucleotides, or at least 25 nucleotides.

The single stranded oligonucleotide probe C may comprise a uniquely specific oligonucleotide probe sequence that is designed to be complementary to sequences of interest present in the target nucleic acid sequence. The probe sequence C can detect the target nucleic acid sequence in a preparation by hybridization of the complementary sequence with the target nucleic acid sequence. In some examples, the target nucleic acid sequence is an aptamer or a signaling polynucleotide (SPN) that is derived from an aptamer. The aptamer comprises a nucleic acid sequence that can specifically bind to an analyte of interest in a sample such as a protein, a DNA or RNA, a sugar or a lipid. The aptamer and/or SPN is used as a detection agent for detection of the presence or absence of the target analyte. The nucleic acid probe sequence may be 100% complementary to the target nucleic acid sequence, or 90%-100%, or 85%-100%, or 80%-100%, or 75% -100%, or 98%, or 97%, or 96%, or 95%, or 90%, or 85%, or 80%, or 75%, or 70%, or 65%, or 60% complementary to the target nucleic acid sequence or a portion thereof. In some examples, the probe sequence may differ from the complementary target sequence by one, two, three, or more nucleotides. In some embodiments, the complementary hybridization between the nucleic acid probe sequence and target sequence will not affect the binding of the target nucleic acid sequence to an analyte (e.g., an allergen). In other examples, the binding of the target nucleic acid sequence to an analyte of interest doesn't affect the complementary hybridization of the nucleic acid probe sequence C.

As used herein, the term “aptamer” refers to a nucleic acid (typically a DNA, RNA or oligonucleotide) that have a high affinity and specificity to a target analyte and comprises 15-100 nucleotides, or about 20-50 nucleotides, or about 20 to 40 nucleotides. For example, an aptamer may comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47,48, 50, 55, 60, 65, 70, 75, 80, 85, 90, 95 or 100 nucleotides. An aptamer has a specific binding affinity to non-nucleic acid or nucleic acid molecules through interactions other than classic Watson-Crick base pairing.

Aptamers can be selected by SELEX (Systematic Evolution of Ligands by Exponential Enrichment), or other in vitro selections of aptamer selection procedures well known in the art. The SELEX method is described in, for example, Gold et al., U.S. Pat. Nos. 5,270,163 and 5,567,588; Fitzwater et al., “A SELEX Primer,” Methods in Enzymology, 267:275-301 (1996); and in Ellington and Szostak, “In Vitro Selection of RNA Molecules that Bind Specific Ligands,” Nature, 346:818-822; the contents of each of which are incorporated herein by reference in their entirety. Aptamers configured to bind to specific target analytes can be selected, for example, by synthesizing an initial heterogeneous library of oligonucleotides, and then selecting oligonucleotides within the library that bind tightly to a particular target analyte. Once an aptamer that binds to a particular target analyte has been identified, it can be replicated using a variety of techniques known in biological and other arts, for example, by cloning and polymerase chain reaction (PCR) amplification followed by transcription.

Target analytes that aptamers can bind to include but are not limited to cells, nucleic acids, small molecules, peptides, proteins and variants thereof, carbohydrates, hormones, sugar, metabolic byproducts, cofactors, drugs and toxins. Aptamers of the present disclosure are preferably specific for a particular analyte. The specificity of the binding is defined in terms of the dissociation constant Kd of the aptamer for its target analyte.

In some embodiments, the nucleic acid probe of the present disclosure further comprises a linker A on one or both ends of the uniquely specific oligonucleotide probe sequence C (FIG. 1). The linker forms an anchor for immobilization to a solid surface. The linker A may comprise a nucleic acid sequence or other molecular moiety or a combination of both. A universal linker can be used. Alternatively, a linker may be specifically designed for each nucleic acid probe. In one preferred embodiment, the linker A is a nucleic acid linker comprising a short oligonucleotide sequence. In this embodiment, the linker sequence is of limited length. For example, the nucleic acid linker may comprise 2-20 nucleotides, or 2-8 nucleotides, or 5-15 nucleotides, or 5-10 nucleotides.

As a non-limiting example, a poly(T) (poly Thymine nucleotides) linker A is added to one end of the uniquely specific oligonucleotide probe sequence C. In some embodiments, a nucleic acid probe may comprise a poly(T)n (n=5-15), a poly(C)n (n=5-10), or a poly(T)n (n=5-10)poly(C)n (n=5-10) linker tagged to the oligonucleotide probe (C in FIG. 1). In some examples, the linker sequence is attached to the 3′ terminus of the probe sequence C. In other examples, the linker sequence is attached to the 5′ terminus of the probe sequence C (FIG. 1). Exemplary linker sequences may include a poly(T)(6) (T6-mer), a poly(T)(7) (T7-mer), a poly(T)(8) (T8-mer), a poly(T)(9) (T9-mer), a poly(T)(10) (T10-mer), a poly(T)(11)(T11-mer), a poly(T)(12) (T12-mer), a poly(T)(13) (T13-mer), a poly(T)(14) (T14-mer), a poly(T)(15) (T15-mer), a poly(T)(10)poly(C)(10), a poly(T)(10)poly(C)(9), a poly(T)(10)poly(C)(8), a poly(T)(10)poly(C)(7), a poly(T)(10)poly(C)(6), a poly(T)(10)poly(C)(5), a poly(T)(5)poly(C)(10), a poly(T)(6)poly(C)(10), a poly(T)(7)poly(C)(10), a poly(T)(8)poly(C)(10), and a poly(T)(9)poly(C)(10). In one embodiment, the linker is a poly(T)(10) (5′TTTTTTTTTT3′; SEQ ID NO. 3).

In some embodiments, the nucleic acid probe may further comprise a spacer B (FIG. 1). The spacer B locates between the linker A and the uniquely specific oligonucleotide probe sequence C (FIG. 1). The spacer is optional.

The spacer B can be any molecule and moiety that provides a physical separation of the linker A from the unique probe sequence C. In some embodiments, the spacer is a short single stranded oligonucleotide, e.g., a DNA spacer or an RNA spacer, or an DNA/RNA hybrid. Spacers of different lengths are characterized and determined for their potential impact on the probe coupling with the solid substrate and its binding with a target nucleic acid sequence (e.g. a SPN). The probe may comprise one or more spacer sequences. The spacer sequence may be of varying lengths. The spacer region may comprise 5-25 nucleotides, or 5-10 nucleotides, or 10-15 nucleotides, or 10-20 nucleotides. In some examples, the spacer sequence may comprise 5, 6, 7, 8, 9, 10, 11, 12, 13, 14, 15, 16, 17, 18, 19, or 20 nucleotides.

In some embodiments, the spacer sequences are of varying nucleotide compositions. Nucleotide compositions of spacer sequences may influence immobilization on a solid surface and hybridization of the uniquely specific probe sequences to their target nucleic acid sequences. The spacer sequence may also be optimized to reduce the tendency of formation of self-dimer and hairpin, therefore, to increase the printing efficiency. The nucleotide compositions of a spacer sequence may be optimized for enhancing attachment of nucleic acid probes to a solid substrate (e.g., a polymer plastic) through simple UV light irradiation. In one preferred embodiment, the optimized spacer sequence will minimize the cross-activity with the control region of a target nucleic acid sequence (e.g., an aptamer and a signaling polynucleotide (SPN)).

The density of immobilized nucleic acid probes on the substrate for different spacer lengths are tested. A possible effect of spacer nucleoside compositions on the hybridization between the uniquely specific probe sequence and target nucleic acid sequence is also investigated. A spacer length with maximal density of probe immobilization and least effect on the hybridization of the two nucleic acids (e.g., SPN and complement probe) is used for optimizing the nucleic acid probe.

In some embodiments, the nucleic acid probe of the present disclosure comprises a spacer sequence selected from the group consisting of SEQ ID Nos.: 4-12, 23-25 and 56-57. In one preferred embodiment, the nucleic acid probe of the present disclosure comprises a spacer sequence presented by SEQ ID NO. 11 (5′GAGAGAGAA3′), or SEQ ID NO. 24 (5′AAGAGAGAG3′).

In one preferred embodiment, the nucleic acid probe to be immobilized is represented by the following formula: a linker (A)-spacer (B)-uniquely specific oligonucleotide probe (C) (FIG. 1), wherein C is a short oligonucleotide probe sequence comprising a sequence complementary to the sequence, or a portion of the sequence of a target nucleic acid molecule and A is a poly (T) linker comprising 5 to 15 T nucleotides and wherein B is a spacer, preferably composed of low contents of A nucleotides. In some embodiments, the spacer sequence is selected from the group consisting of sequences presented by SEQ ID Nos.: 4-12, 23-25 and 56-57. In one embodiment, the poly(T) linker and spacer sequence may be tagged at the 5′ end of the probe. In another embodiment, the poly(T) linker and spacer sequence may be tagged at the 3′ end of the probe.

In some embodiments, the present nucleic acid probes are synthesized, PCR amplified or recombinantly constructed prior to deposition on the substrate surface.

Tables 2 and 9 show a list of nucleic acid probe sequences and specific linker sequences and spacer sequences for aptamers that can specifically bind to peanut allergen. As a non-limiting example, a nucleic acid probe that can capture the SPN (SEQ ID NO. 1) specific to peanut allergen AraHl may comprise a uniquely specific oligonucleotide sequence, i.e., SEQ ID NO. 2 that is complementary to the sequence of SEQ ID NO. 1. In some examples, the nucleic acid probe for AraH1 SPN comprises a nucleic acid sequence selected from the group consisting of SEQ ID Nos. 13 to 21. In one preferred embodiment, the nucleic acid probe for capturing AraHl SPN comprises 5′ TTTTTTTTTTGAGAGAGAATTCGCACACA 3′ (SEQ ID NO. 20).

The present disclosure provides nucleic acid probes that can capture a signaling polynucleotide that binds to peanut (i.e., PC60). In some examples, the nucleic acid probe comprises a uniquely specific oligonucleotide sequence: 5′ TCAAGTGGTCAT3′ (SEQ ID NO. 55) that is complementary the sequence of PC60 (5′ TAGGGAAGAGAAGGACATATGATCGTACCGCAAGTGACGTGTCCGTGCCGTGAT TGACTAGTACATGACCACTTGA3′; SEQ ID NO. 54). In some examples, the nucleic acid probe comprises a sequence selected from the group consisting of SEQ ID Nos. 58 to 63.

In accordance with the present disclosure, a target capturing probe may be used in combination with a control probe for detection of an analyte of interest in a sample, e.g., an allergen in a sample. The control probe is optimized for direct immobilization to a solid substrate by UV light irradiation. In some embodiments, the control probe may comprise a similar formula shown in FIG. 1. The control probe will comprise a linker sequence (A), a spacer (B) and a control probe (C) that does not specifically bind to the target sequence. As a non-limiting example, control probes can be used to measure a total protein in a detection assay for normalizing the detection signal.

In some examples, a control probe may comprise a sequence selected from the group consisting of SEQ ID Nos. 26-39 and 47-53. In one preferred embodiment, the control probe will comprise a sequence of SEQ ID NO. 31 (5′ CCCCCCCGGTAAGAGAGAGTTTTTTTTTT3′), or a sequence of SEQ ID NO. 38 (5′CCCCCGGTAAGAGAGAG TTTTTTTTTT3′).

In one aspect of the present disclosure, a kit for detecting an analyte in a sample is provided. The kit comprises a signaling polynucleotide (SPN) that specifically binds to the target analyte, a nucleic acid probe that comprises a uniquely specific oligonucleotide complementary to the sequence or a portion of the sequence of the SPN, and a control probe for measuring an internal control signal (e.g., a total protein from the sample). The nucleic acid probe will capture the SPN through hybridization between the complementary sequences. In some embodiments, the nucleic acid probe to the SPN and the control probe are optimized to comprise a linker sequence and a spacer sequence. The optimized probes are immobilized to a solid substrate through the linker sequence using UV light irradiation.

### 2. Nucleic Acid Chips

In another aspect, the present disclosure provides nucleic acid chips comprising solid substrates and nucleic acid probes immobilized thereto. The substrate may be any solid substrate such as a glass chip, a plastic and a resin. The substrate with immobilized nucleic acid probes may be used as sensors for detection an analyte of interest in a sample. Nucleic acid chips may be integrated with any detection devices and microfluidic systems. In some examples, the nucleic acid chip can be obtained by supplying a nucleic acid probe on a predetermined position on the substrate and immobilizing the probe thereon.

The material of the substrate is not particularly limited. Examples of the substrate include but are not limited to, flat substrates such as a glass substrate, a plastic substrate, and a silicon wafer; a three-dimensional structure having an irregular surface; a spherical body such as a bead; and rod-, cord-, and thread-shaped structures. The surface of the substrate may be processed such that a probe can be immobilized thereon. In particular, a substrate prepared by introducing a functional group to its surface to make chemical reaction possible. In one preferred embodiment, the substrate is a flat chip. The shape and size of the substrate is not particularly limited.

In some embodiments, the substrate is made of polymeric materials. Polymers are of particular interest since plastics are of low-cost and amenable to high volume manufacturing processes. Exemplary polymers as potential solid supports for nucleic acid chip production including polyethylene (PE), polypropylene (PP), polyvinyl chloride (PVC), polystyrene (PS), cyclic olefin copolymer (COC), poly (methyl methacrylate) (PMMA), poly(dimethylsiloxane) (PDMS), polycarbonate (PC), nylon, polytetrafluoroehylene (Teflon), and polystyrene and poly(ethylene terephthalate) (PET).

Nucleic acid probes can be immobilized on one or more predetermined discrete areas on the substrate. Each discrete area may comprise a plurality of spots. Each spot on the substrate contains multiple identical nucleic acid molecules. In some embodiments, a spot pitch of 100 μm to 500 μm on a substrate may be achieved. As non-limiting examples, the probes can be spotted at a pitch of about 100 μm, about 120 μm, about 150 μm, about 180 μm, about 200 μm, about 220 μm, about 240 μm, about 260 μm, about 280 μm, about 300 μm, about 350 μm, about 400 μm, about 450 μm, or about 500μm. In some embodiments, a discrete area comprises 2 to 1000, or 10 to 1000, or 200 to 1000, or 500 to 1000, or at least 2, or at least about 3, at least 4, at least 5, or at least 100, or at least 200, or at least 500, or at least 1000, or more isolated spots. In some embodiments, the amount of immobilized probes per immobilization location (spot) for each probe can vary from one to another. In some embodiments, a variety of nucleic acid probes are immobilized in a definite pattern on the surface. The probes may be arrayed in parallel and/or in a constant and definite order. In some examples, two or more nucleic acid probes specific to different target sequences are spotted on discrete areas on the substrate.

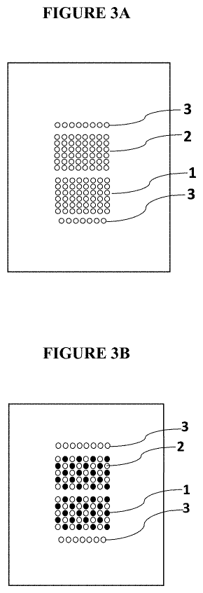

In one preferred embodiment, a solid substrate may comprise a discrete area with a nucleic acid probe specific to a target nucleic acid sequence immobilized to, which is referred to as a reaction panel and a discrete area with a control probe which is referred to as a control panel. FIG. 3A illustrated an exemplary pattern of the reaction panel (e.g., 1 in FIG. 3A) and control panel (e.g., 2 in FIG. 3A) of a nucleic acid chip. In another embodiment, the reaction panels (1 in FIG. 3B) with nucleic acid probes and control panels (2 in FIG. 3B) with control sequences are positioned in a checkerboard pattern on the substrate (FIG. 3B). The checkerboard pattern of the probe spots may minimize optical alignment variability.

Other spatial patterns of spots on the substrate comprising different nucleic acid probes and control sequences may be included. A simple digital pattern may be made with a plurality of spots with each spot having a set of nucleic acid probes and a set of control sequence, to make a binary code (0=control sequence, 1=nucleic acid probes). The chip may be divided into a plurality of reaction sites; each site comprises multiple spots.

As a non-limiting example, a nucleic acid chip may be a chipannel made of a polymeric material. The chipannel will comprise a nucleic acid probe that can capture an aptamer or a SPN that binds to a target analyte. The chipannel comprises a nucleic acid probe that can capture AraHl SPN (SEQ ID NO. 1) specific to peanut allergen. The nucleic acid probe comprises a linker sequence, a spacer sequence and a uniquely specific oligonucleotide sequence that is complementary to the sequence of a portion of the sequence of AraHl SPN (SEQ ID NO. 1). The linker sequence is a poly(T) sequence, e.g., SEQ ID NO. 3. The spacer sequence may be selected from the group consisting of SEQ ID Nos. 4-12, 23-25 and 56-57. In one embodiment, the nucleic acid probe comprises a uniquely specific oligonucleotide sequence of SEQ ID NO. 2. In some examples, the nucleic acid probe comprises a nucleic acid sequence selected from the group consisting of SEQ ID Nos. 13-21. The nucleic chip is further spotted with a control probe, wherein the control sequence is selected from the group consisting of SEQ ID Nos. 26-39 and 47-53.

In some examples, the chipannel may further comprise one or more panels with fiducial probes immobilized thereto. As used herein, the term “fiducial probe” means a fiducial marker placed in the field of view for an imaging system, for use as a point of reference or a measure. In some embodiments, the fiducial probe is an oligonucleotide labeled with a fluorophore. The fiducial probe may be selected from the group consisting of SEQ ID Nos. 64-66. The fiduciary spots can guide image processing by an imaging mechanism (e.g., a camera) of a detector module. In some examples, the chipannel may further comprise a plurality of fluidic channels configured to transport fluids in and out from the probe panels.

The probe panels (e.g., reaction panel and control panel) and fiducial panel may be arranged in a pattern as illustrated in FIGS. 3A and 3B.

Surface oligonucleotide density is crucial for a wide variety of applications of DNA chips. The hybridization of complementary strands between a probe and a target nucleic acid sequence is strongly dependent on surface oligonucleotide density, e.g., the thermodynamic stability of double-stranded nucleic acid. Surface oligonucleotide density could also affect the kinetics of target/probe hybridization. According to the present disclosure, the density of nucleic acid sequences on a chipannel is optimized to ensure the best sensitivity and specificity of the probes to their target sequences and binding affinity for target analyte-SPN recognition, and to meet the requirements for optical detection during a detection assay (e.g. signal intensity and background).

In some embodiments, nucleic acid chips can be configured for use with an analytical device. As used herein, the term “analytic device” generally refers to an arbitrary device configured for conducting at least one analysis, specifically one detection analysis. The analytic device therefore generally may be an arbitrary device configured for performing at least one allergen detection purpose. Specifically, the analytic device may be capable of performing at least one detection of the at least one allergen in a food sample, e.g., the presence and/or absence of the food sample in the sample. The analytic device therefore generally may be an arbitrary device configured for performing at least one diagnostic purpose, e.g., allergic reaction. As non-limiting examples, the oligonucleotide chips (e.g., chipannels) may be used in a detection device discussed in the PCT Patent Application Publication Nos. WO2015066027, WO2016149253, WO2017160616, and WO2018156535; the contents of each of which are incorporated herein by reference in their entirety. In other examples, the oligonucleotide chips (e.g., chipannels) may be used in the analytic cartridge as discussed in Applicant's pending PCT Patent Application No. PCT/US2019/018860 and U.S. Provisional Patent Application No. 62/741,756; the contents of each of which are incorporated herein by reference in their entirety.

### 3. Chip fabrication

Various technologies and methods can be used for immobilization of nucleic acid probes to a carrier such as a solid substrate. The major methods used to immobilize nucleic acids on a solid substrate include (a) synthesis of nucleic acid probes directly on the surface of the substrate (e.g., Oligonucleotide array manufactured by Affymetrix Co., Ltd.), and (b) deposition and immobilization of pre-synthesized nucleic acids on functionalized substrates (e.g., glass slides an plastic plates).

Pres-synthesized nucleic acids can be immobilized to the substrate through a chemical bonding (e.g., covalent bonding), and absorption. Well-known adsorption methods include embedding, co-adsorption, and substitution. Chemical reaction-based attachment often requires complicated chemical modifications of both the nucleic acid probes and the surface. A number of treatment steps are performed for immobilizing the probes to a substrate, e.g., a polymer plastic. For example, the surface of a substrate may be pre-treated to improve the immobilization of nucleic acid probes, including introducing a functional group (e.g., an amino group, a thiol group, a mercapto group (—SH), a sulfonato group (—SO3-), and a carboxyl group (—COOH)) to the surface of the substrate. DNA oligonucleotide probes can also be immobilized to non-modified plastic substrates through SN₂ reaction (e.g., Fixe et al., One-step immobilization of aminated and thiolated DNA onto poly(methylmethacrylate) (PMMA) substrates; Lab Chip. 2004 June; 4(3):191-195), binding buffers (e.g., Liu and Rauch, DNA probe attachment on plastic surfaces and microfluidic hybridization array channel devices with sample oscillation; Anal Biochem. 2003 Jun. 1; 317(1):76-84) or direct attachment by UV exposure (e.g., Sabourin et al., Microfluidic DNA microarrays in PMMA chips: streamlined fabrication via simultaneous DNA immobilization and bonding activation by brief UV exposure, Microdevices, 2010; 12:673-681). Li et al., irradiated PC with UV/ozone to facilitate the attachment of amino-modified DNA probes (Li et al., DNA detection on plastic: surface activation protocol to convert polycarbonate substrates to biochip platforms, Anal Chem. 2007; 79:426-433). Kimura et al. reported UV-induced attachment of DNA strands modified with poly(dT) and an undisclosed linker sequence, to PC, PMMA, and PET (Kimura et al., One-step immobilization of poly(dT)-modified DNA onto non-modified plastic substrates by UV irradiation for microarrays, Biochem Biophys Res Commun. 2006; 347: 477-484).

Previous studies have demonstrated that UV irradiation could successfully convert inert plastics into bio-reactive substrates for nucleic acid immobilization/hybridization. However, chemical modifications, e.g., amino modification by Li et al. make DNA probe more expensive. Nucleic acid probes of the present disclosure are optimized, comprising a linker sequence and a spacer sequence for direct UV mediated attachment to unmodified polymer surfaces.

In accordance with the present disclosure, a simple method of immobilizing the nucleic acid probe as disclosed herein to a solid substrate (e.g., a polymer plastic) is provided; the method involves simple UV irradiation used to directly immobilize linker/spacer sequences tagged oligonucleotide probes to many different types of plastics without any surface modification. The one-step, cost-effective DNA-linking method on non-modified polymers significantly simplifies chip fabrication procedures and permits great flexibility to plastic material selection, thus making it convenient to integrate nucleic acid chips into plastic detection systems (e.g., a plastic analytical cartridge and a plastic microfluidic system). The method offers higher immobilization as well as high hybridization efficiency.

In some embodiments, the nucleic acid probes represented by the general formula of FIG. 1 is immobilized to a solid substrate via UV light cross-linking at an exposing wavelength of about 300 nm to 500 nm, preferably at an exposing wavelength of 350 nm. The substrate may comprise a super-hydrophobic polymeric surface.

Typically, the induced interaction between the probes and the solid substrate is a covalent binding of the nucleic acid to the material. Crosslinking by light in the range of about 300 nm to about 500 nm may, for example, be carried out by using near or long wave UV light, UVA light or black light. The term “range of about 300 nm to about 500 nm” refers to every single wavelength between 300 nm and 500 nm. It preferably also refers to certain subranges thereof, e.g. a subrange of 300 to 320 nm, 320 to 340 nm, 340 to 360nm, 360 to 380 nm, 380 to 400 nm, 400 to 420 nm, 420 to 440 nm, 440 to 460 nm, 460 to 480 nm, 480 to 500 nm. The wavelength of the light to be used may be determined primarily by the choice of lamps. For instance, in order to establish a wavelength in the spectrum of 300 to 500 nm a high-pressure mercury UV-lamp may be used. Such a lamp typically emits not only one wavelength, but a spectrum of wavelengths, as the person skilled in the art would know. The term “spectrum of 300-500 nm” relates to such a typical spectrum emitted from a high-pressure mercury UV-lamp. Alternatively, the light may also be emitted from a LED, which may have a different emission spectrum or from any other lamp or light source known to the person skilled in the art as long the majority of the emitted wavelengths are within the range of 300 to 500 nm.

In some embodiments, optimized spotting buffers and wash buffers are developed and used for immobilizing nucleic acid probes to the substrate, therefore, to improve UV printing efficiency of probes and washing efficiency. Additionally, wash buffers are tested for removing access probes that are not immobilized onto the solid substrate and any debris from the process.

Nucleic acid probes are diluted in a buffer, e.g., a sodium phosphate buffer containing Triton X to a desired final concentration of the probes. Spotting may be performed using any commercially available pin-spotting systems, inkjet systems, micro contact printing; photochemical or photolithographic methods or the like. After spotting, the spots on the substrate are allowed to dry and then exposed to UV irradiation at a pre-determined light wavelength. The power and exposure time are tested and determined as well. Subsequently, the plastic substrate is washed under agitation using wash buffers, e.g., standard saline citrate (SSC) buffer or optimized wash buffers.

During the immobilization, surface oligonucleotide density is controlled by varying immobilization conditions, including but not limiting to pre-synthesized DNA strand concentration, solution ionic strength, spotting buffer concentration, interfacial electrostatic potential, whether duplex or single stranded oligonucleotides are used, and reaction time.

### Applications

The present disclosure provides a detection method comprising imparting a sample which is suspected of containing an analyte of interest, to be detected with the present nucleic acid probes and chips as disclosed herein, and detecting the presence or absence of the analyte of interest in the sample.

The detection assay and method for detecting an analyte of interest in a sample comprising (a) providing a complex formed from (i) a sample suspected of containing the analyte of interest and (ii) a nucleic acid based detection agent in a condition allowing the binding of the analyte to the detection agent, wherein the detection agent comprises a nucleic acid sequence that binds to the analyte of interest; (b) contacting the complex of the analyte of interest and the detection agent to a nucleic acid probe immobilized to a solid substrate, wherein the probe comprises an oligonucleotide probe sequence that is complementary to the sequence or a portion of the sequence of the detection agent; (c) applying a detection module to the solid substrate for detecting a signal from the detection agent and the oligonucleotide probe, wherein if the analyte is not present in the sample, the detection agent not bound to the analyte is coupled to the solid substrate via the direct hybridization between the probe sequence and the target sequence of the detection agent; and (d) measuring the amount of the detection agent wherein the amount of the detection agent indicates where or not the analyte of interest is present in the sample. In some aspects, the substrate is a nucleic acid chip (e.g., a chipannel).

In some embodiments, the detection assay and method for detecting an analyte of interest in a sample comprising the steps (1) immobilizing a nucleic acid probe consisting of a linker (A)—a spacer (B)—a probe sequence (C) at discrete locations on a solid substrate so as to fabricate a chipannel; (2) reacting a sample suspected of containing the analyte of interest with a target nucleic acid (e.g., a SPN) based detection agent so as to prepare analyte of interest:detection agent complexes; (3) reacting the analyte of interest:detection agent complexes with the chipannel in step (1); and (4) detecting the presence and absence of the analyte of interest in the sample.

In some embodiments, the target nucleic acid sequence is an aptamer or a signaling polynucleotide (SPN) that binds to the analyte. In some examples, the aptamer or SPN comprises a detectable marker. Detectable markers may include radioisotopes, fluorophores, chromophores, enzymes, dyes, metal ions, ligands, biotin, avidin, streptavidin and haptens, quantum dots, polyhistidine tags, Myc tags, Flag tags, human influenza hemagglutinin (HA) tags and the like.

In one preferred embodiment, the aptamer or SPN is labeled with a fluorophore. As non-limiting examples, a fluorophore may include but is not limited to, derivatives of boron-dipyrromethene (BODIPY e.g., BODIPY TMR dye; BODIPY FL dye), Fluorescein including derivatives thereof, Rhodamine including derivatives thereof, Dansyls including derivatives thereof (e.g. dansyl cadaverine), Texas red, Eosin, Cyanine dyes, Indocarbocyanine, Oxacarbocyanine, Thiacarbocyanine, Merocyanine, Squaraines and derivatives Seta, SeTau, and Square dyes, Naphthalene and derivatives thereof, Coumarin and derivatives thereof, Pyridyloxazole, Nitrobenzoxadiazole, Benzoxadiazole, Anthraquinones, Pyrene and derivatives thereof, Oxazine and derivatives, Nile red, Nile blue, Cresyl violet, Oxazine 170, Proflavin, Acridine orange, Acridine yellow, Auramine, Crystal violet, Malachite green, Porphin, Phthalocyanine, Bilirubin, Tetramethylrhodamine, Hydroxycoumarin, Aminocoumarin; Methoxycoumarin, Cascade Blue, Pacific Blue, Pacific Orange, NBD, R-Phycoerythrin (PE), Red 613; PerCP, TruRed; FluorX, Cy2, Cy3, Cy5 and Cy7, TRITC, X-Rhodamine, Lissamine Rhodamine B, Allophycocyanin (APC) and Alexa Fluor dyes (e.g., Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 635, Alexa Fluor 647, Alexa Fluor 660, Alexa Fluor 680, Alexa Fluor 700 and Alexa Fluor 750).

The analyte of interest may be selected from bacteria, fungi, virus, cell lines, tissues, proteins, nucleic acids, carbohydrates, lipids, polysaccharides, glycoproteins, hormones, receptors, antigens, antibodies, and enzymes. In one preferred embodiment, the analyte of interest is an allergen, such as a food allergen (e.g., peanut, milk, egg white, fish, sea food, wheat and tree nuts). In other embodiments, the analyte may be a pathogen. As used herein, the term “pathogen” means any disease-producing agent (especially a virus or bacterium or other microorganism). In other embodiments, the target analyte may be a disease associated protein to diagnose, stage diseases and disorders. Disease associated proteins may be secreted polypeptides and peptides (e.g. circulating molecules); cell surface proteins (e.g. receptors); biomarkers that are expressed or overexpressed in a particular disease condition; isoforms, derivatives and/or variants of a particular protein that are only present in a disease condition; mutated proteins that cause a disorder; antibodies (e.g., IgE associated with allergic reaction); and proteins derived from another organism which causes a clinical condition in the host such as viral infection.

In some embodiments, detection assays can be carried out using a chipannel that includes a first area that contains a nucleic acid probe having a uniquely specific nucleotide sequence complementary to a target nucleic acid molecule and a second area that contains a control oligonucleotide probe that does not hybridize to the target nucleic acid molecule. The control probe measures a total signal from the sample to provide an internal control. In some examples, the chipannel comprises a plurality of the first areas containing the nucleic acid probe and a plurality of the second areas containing the control probe wherein the plurality of the first areas and the plurality of the second areas are positioned with a checkerboard pattern on the chipannel.

In some embodiments, the chipannel is configured as an integrated part of an analytic cartridge that is configured for processing a sample suspected of containing a target analyte and reacting the target analyte with a nucleic acid based detection agent in a condition allowing formation of the target analyte: detection agent complexes.

In some embodiments, detection assays of the present invention further comprising washing the chipannel after the reaction. A wash solution may be used to wash the surface of the chipannel simply and uniformly. A suitable washing method may vary depending on the kind of the substrate. For example, in the case where glass is used as a substrate, there may be mentioned of a method in which a surface of a substrate is sufficiently washed with an aqueous solution of sodium hydroxide having a predetermined concentration to remove contaminants attached on the substrate.

In some embodiments, a detection module may be used for detecting and measuring a fluorescence signal from the hybridization reaction between the probe and the detection agent. In some examples, the detection module is an imaging system (e.g., a camera), or an electronic light detector employed for DNA chip analysis.

### Equivalents and Scope

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

A number of possible alternative features are introduced during the course of this description. It is to be understood that, according to the knowledge and judgment of persons skilled in the art, such alternative features may be substituted in various combinations to arrive at different embodiments of the present disclosure.

Any patent, publication, internet site, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

### EXAMPLES

### Example 1: Characterization of Modified DNA Probes Specific to AraH1 SPN on a COP Plastic

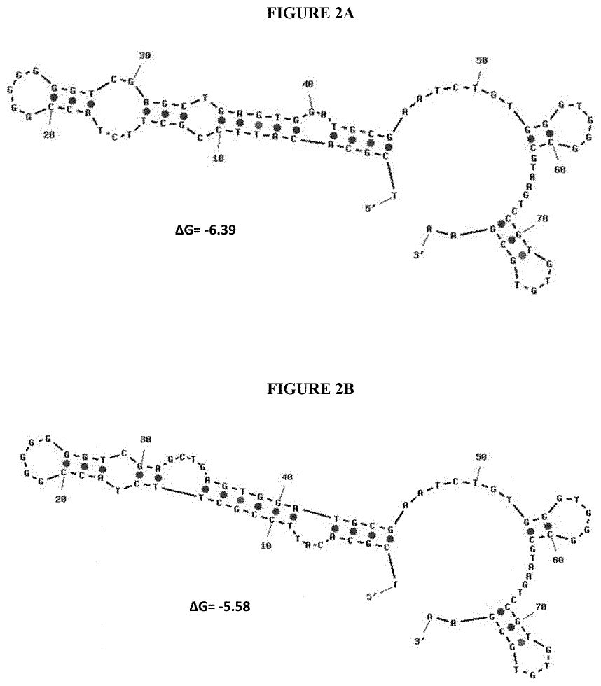

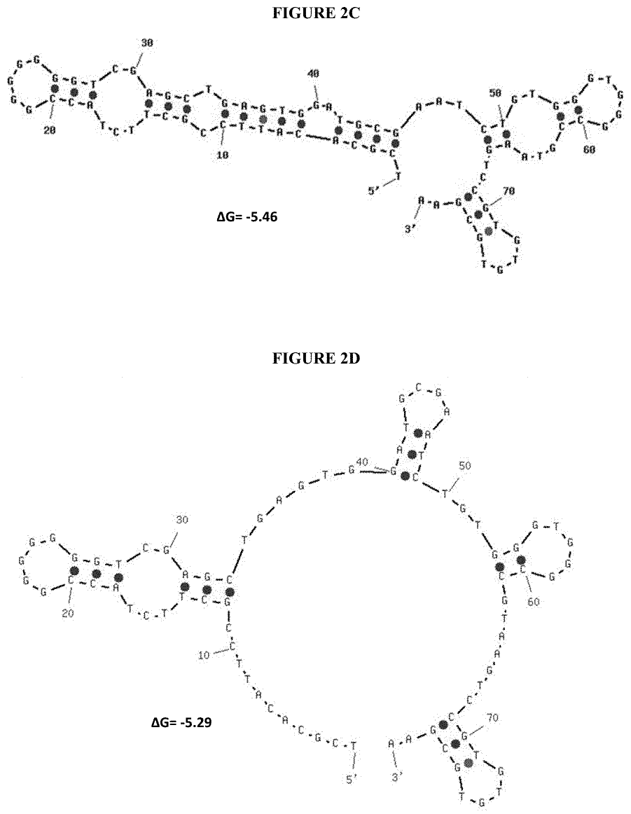

A uniquely specific nucleic acid probe sequence (5′TTCGCACACA 3′, SEQ ID NO. 2) was used to generate optimized probes for hybridizing/capturing a target nucleic acid sequence, i.e., a signaling polynucleotide (SPN) (5′ TCGCACATTCCGCTTCTACCGGGGGGGTCGAGCTGAGTGGATGCGAATCTGTGGG TGGGCCGTAAGTCCGTGTGTGCGAA3′; SEQ ID NO. 1) (FIG. 2). The SPN can specifically bind to a peanut allergen AraHl (referred to as AraHl SPN). The secondary structures of the SPN are illustrated in FIGS. 2A to 2D and their parameters in each condition are in Table 1.

TABLE 1
Structural characters of AraH1 SPN (SEQ ID NO. 1)

| | ΔG | | ΔH | ΔS |
|---|---|---|---|---|
| AraH1 | (kcal · | Tm | (kcal · | (cal · K − 1 |
| SPN | mole⁻¹) | (° C.) | mole⁻¹) | mole⁻¹) |
| FIG. 2A | −6.39 | 39.4 | −138.3 | −442.43 |
| FIG. 2B | −5.58 | 38.6 | −128.2 | −411.27 |
| FIG. 2C | −5.46 | 35.9 | −154.4 | −499.54 |
| FIG. 2D | −5.29 | 38.5 | −121.7 | −390.45 |

The uniquely specific nucleic acid probe sequence SEQ ID NO. 2 is complementary to the sequence AraHl SPN (positions 71 to 80 of SEQ ID NO. 1).

The uniquely specific nucleic acid probe sequence (SEQ ID NO. 2) was modified at the 5′end by adding a poly(T)(10) linker sequence (5′TTTTTTTTTT 3′; SEQ ID NO. 3) and a variety of spacer sequences to facilitate the attachment of the oligonucleotides to the solid substrate. The modified oligonucleotide probes that are immobilized to a polymeric plastic then include a UV printing linker sequence (SEQ ID NO. 3), a spacer sequence (e.g., selected from Table 2) and a uniquely specific oligonucleotide probe (SEQ ID NO. 2) that is complementary to the target nucleic acid molecule, i.e. AraH1 SPN (SEQ ID NO. 1). These modified probes will be used as detection probes to recognize the SPN. Nine modified probes are listed in Table 2. A probe having a classical UV-linkable oligonucleotide linker comprising poly(T)(10)poly(C)(10)poly(A)(5) (5′ TTTTTTTTTTCCCCCCCCCCAAAAATTCGCACACA 3′, SEQ ID NO. 13) was used for comparison.

TABLE 2
Revisions of optimized probe sequences
for AraH1 SPN (SEQ ID NO. 1)

| AraH1 | | | AraH1 | Optimized |
|---|---|---|---|---|
| SPN | | | probe | probe |
| probe | Linker | Spacer | se- | sequence |
| revision | (5′-3′) | sequence | quence | (5′-3′) |
| Rev1 | TTTT | CCCC | TTCG | TTTT |
| | TTTT | CCCC | CACA | TTTT |
| | TT | CCAA | CA | TTCC |
| | (SEQ ID | AAA | (SEQ ID | CCCC |
| | NO. 3) | (SEQ ID | NO. 2) | CCCC |
| | | NO. 4) | | AAAA |
| | | | | ATTC |
| | | | | GCAC |
| | | | | ACA |
| | | | | (SEQ ID |
| | | | | NO. 13) |
| Rev2 | TTTT | CCCC | TTCG | TTTT |
| | TTTT | CCCC | CACA | TTTT |
| | TT | CC | CA | TTCC |
| | (SEQ ID | (SEQ ID | (SEQ ID | CCCC |
| | NO. 3) | NO. 5) | NO. 2) | CCCC |
| | | | | TTCG |
| | | | | CACA |
| | | | | CA |
| | | | | (SEQ ID |
| | | | | NO. 14) |
| Rev3 | TTTT | CCAA | TTCG | TTTT |
| | TTTT | CACA | CACA | TTTT |
| | TT | AC | CA | TTCC |
| | (SEQ ID | (SEQ ID | (SEQ ID | AACA |
| | NO. 3) | NO. 6) | NO. 2) | CAAC |
| | | | | TTCG |
| | | | | CACA |
| | | | | CA |
| | | | | (SEQ ID |
| | | | | NO. 15) |
| Rev4 | TTTT | CCAA | TTCG | TTTT |
| | TTTT | CCAA | CACA | TTTT |
| | TT | CC | CA | TTCC |
| | (SEQ ID | (SEQ ID | (SEQ ID | AACC |
| | NO. 3) | NO. 7) | NO. 2) | AACC |
| | | | | TTCG |
| | | | | CACA |
| | | | | CA |
| | | | | (SEQ ID |
| | | | | NO. 16) |
| Rev5 | TTTT | CC | TTCG | TTTT |
| | TTTT | (SEQ ID | CACA | TTTT |
| | TT | NO. 8) | CA | TTCC |
| | (SEQ ID | | (SEQ ID | TTCG |
| | NO. 3) | | NO. 2) | CACA |
| | | | | CA |
| | | | | (SEQ ID |
| | | | | NO. 17) |
| Rev6 | TTTT | AAAA | TTCG | TTTT |
| | TTTT | A | CACA | TTTT |
| | TT | (SEQ ID | CA | TTAA |
| | (SEQ ID | NO. 9) | (SEQ ID | AAAT |
| | NO. 3) | | NO. 2) | TCGC |
| | | | | ACAC |
| | | | | A |
| | | | | (SEQ ID |
| | | | | NO. 18) |
| Rev7 | TTTT | GGAA | TTCG | TTTT |
| | TTTT | GGAA | CACA | TTTT |
| | TT | A | CA | TTGG |
| | (SEQ ID | (SEQ ID | (SEQ ID | AAGG |
| | NO. 3) | NO. 10) | NO. 2) | AAAT |
| | | | | TCGC |
| | | | | ACAC |
| | | | | A |
| | | | | (SEQ ID |
| | | | | NO. 19) |
| Rev8 | TTTT | GAGA | TTCG | TTTT |
| | TTTT | GAGA | CACA | TTTT |
| | TT | A | CA | TTGA |
| | (SEQ ID | (SEQ ID | (SEQ ID | GAGA |
| | NO. 3) | NO. 11) | NO. 2) | GAAT |
| | | | | TCGC |
| | | | | ACAC |
| | | | | A |
| | | | | (SEQ ID |
| | | | | NO. 20) |
| Rev9 | TTTT | GAGA | TTCG | TTTT |
| | TTTT | GAGA | CACA | TTTT |
| | TT | AA | CA | TTGA |
| | (SEQ ID | (SEQ ID | (SEQ ID | GAGA |
| | NO. 3) | NO. 12) | NO. 2) | GAAA |
| | | | | TTCG |
| | | | | CACA |
| | | | | CA |
| | | | | (SEQ ID |
| | | | | NO. 21) |
| Rev12 | TTTT | GAGA | TTCG | TTTT |
| | TTTT | GAGA | CACA | TTTT |
| | TT | A | CACG | TTGA |
| | (SEQ ID | (SEQ ID | G | GAGA |
| | NO. 3) | NO. 11) | (SEQ ID | GAAT |
| | | | NO. 69) | TCGC |
| | | | | ACAC |
| | | | | ACGG |
| | | | | (SEQ ID |
| | | | | NO. 70) |

TABLE 3
Characterization of nucleic acid probes specific
to AraH 1 SPN (SEQ ID NO. 1) for UV cross-linking

| AraH 1 | Self-dimer | Hairpin | To AraH1 -1^{st} | To AraH1 -2^{nd} |
|---|---|---|---|---|
| SPN probe | (Kcal/mol, | Tm | (Kcal/mol, | (Kcal/mol, |
| revision | 50 mM Na) | (° C.) | 50 mM Na) | 50 mM Na) |
| Rev1 | −7.78 | 22.4 | −18.82 | −18.42 |
| Rev2 | −3.61 | No stable | −18.82 | −18.42 |
| | | hairpin | | |
| Rev3 | −3.61 | −41.1 | −18.82 | −12.22 |
| Rev4 | −3.61 | −41.1 | −18.82 | −12.22 |
| Rev5 | −3.16 | No stable | −18.82 | −12.22 |
| | | hairpin | | |
| Rev6 | −16.52 | 35.9 | −18.82 | −13.7 |
| Rev7 | −5.36 | 27.8 | −18.82 | −13.7 |
| Rev8 | −8.51 | 15.3 | −18.82 | −13.7 |
| Rev9 | −5.36 | 9.6 | −18.82 | −13.7 |

The probes were analyzed for self-dimers and hairpins. The results are shown in Table 3. These probes were also tested for interacting with AraH1 SPN (SEQ ID NO.1) and the effects on the interaction of SPN with its target allergen AraHl.

A control oligonucleotide (5′CCCCCCCGGT3′; SEQ ID NO. 22) was modified to develop a control probe. The control probe will be used together with detection probes specific to AraH1 SPN (as shown in Table 2). For example, the control probe will be immobilized to a control area of a chipannel that comprises nucleic acid probe specific to the SPN of SEQ ID NO. 1. The control oligonucleotide (SEQ ID NO. 22) was modified at the 3′end by adding a poly(T)(10) linker sequence (5′TTTTTTTTTT 3′; SEQ ID NO. 3) and a variety of spacer sequences (e.g., selected from Table 4) between the control oligonucleotide and the linker sequence to facilitate the attachment of the oligonucleotide to the solid substrate.

The initial tests indicate that the spacer sequence 5′AAGAGAGAG3′ (SEQ ID NO. 24) increases the efficiency of UV cross-linking to a plastic chip. Different control oligonucleotides (SEQ ID Nos. 40-46; Table 5) were designed and tested. Table 5 lists other 7 control probes that include a spacer sequence of SEQ ID NO. 24 and a poly(T)(10) linker sequence (SEQ ID NO. 3) at the 3′ end of the oligonucleotide.

TABLE 4
Control probe sequences with
a 3′-end linker sequence

| | 5′G | | | Control |
|---|---|---|---|---|
| Control | control | | | probe |
| probe | probe | Spacer | Linker | sequence |
| revision | (5′-3′) | sequence | (5′-3′) | (5′-3′) |
| 3′-5′G | CCCCC | AAAAA | TTTTT | CCCCC |
| Rev1 | CCGGT | (SEQID | TTTTT | CCGGT |
| | (SEQ ID | NO. 9) | (SEQ ID | AAAAA |
| | NO. 22) | | NO. 3) | TTTTT |
| | | | | TTTTT |
| | | | | (SEQ ID |
| | | | | NO. 26) |
| 3′-5′G | CCCCC | N/A | TTTTT | CCCCC |
| Rev2 | CCGGT | | TTTTT | CCGGT |
| | (SEQ ID | | (SEQ ID | TTTTT |
| | NO. 22) | | NO. 3) | TTTTT |
| | | | | (SEQ ID |
| | | | | NO. 27) |
| 3′-5′G | CCCCC | CCAAC | TTTTT | CCCCC |
| Rev3 | CCGGT | CAACC | TTTTT | CCGGT |
| | (SEQ ID | (SEQ ID | (SEQ ID | CCAAC |
| | NO. 22) | NO. 7) | NO. 3) | CAACC |
| | | | | TTTTT |
| | | | | TTTTT |
| | | | | (SEQ ID |
| | | | | NO. 28) |
| 3′-5′G | CCCCC | CCCCC | TTTTT | CCCCC |
| Rev4 | CCGGT | CCCCC | TTTTT | CCGGT |
| | (SEQ ID | (SEQ ID | (SEQ ID | CCCCC |
| | NO. 22) | NO. 5) | NO. 3) | CCCCC |
| | | | | TTTTT |
| | | | | TTTTT |
| | | | | (SEQ ID |
| | | | | NO. 29) |
| 3′-5′G | CCCCC | AAAGG | TTTTT | CCCCC |
| Rev5 | CCGGT | AAGG(SEQ ID | TTTTT | CCGGT |
| | (SEQ ID | NO. 23) | (SEQ ID | AAAGG |
| | NO. 22) | | NO. 3) | AAGGT |
| | | | | TTTTT |
| | | | | TTTT |
| | | | | (SEQ ID |
| | | | | NO. 30) |
| 3′-5′G | CCCCC | AAGAG | TTTTT | CCCCC |
| Rev6 | CCGGT | AGAG(SEQ ID | TTTTT | CCGGT |
| | (SEQ ID | NO. 24) | (SEQ ID | AAGAG |
| | NO. 22) | | NO. 3) | AGAGT |
| | | | | TTTTT |
| | | | | TTTT |
| | | | | (SEQ ID |
| | | | | NO. 31) |
| 3′-5′G | CCCCC | AAAGA | TTTTT | CCCCC |
| Rev7 | CCGGT | GAGAG | TTTTT | CCGGT |
| | (SEQ ID | (SEQ ID | (SEQ ID | AAAGA |
| | NO. 22) | NO.25) | NO. 3) | GAGAG |
| | | | | TTTTT |
| | | | | TTTTT |
| | | | | (SEQ ID |
| | | | | NO. 32) |

TABLE 5
Control probe sequences with a
3′-end linker sequence

| | 5′G | | | Control |
|---|---|---|---|---|
| Control | control | | | probe |
| probe | probe | Spacer | Linker | sequence |
| version | (5′-3′) | (5′-3′) | (5′-3′) | (5′-3′) |
| 3′-5′G | CACCC | AAGA | TTTTT | CACCCG |
| Rev 8 | GGTA | GAGA | TTTTT | GTAGAA |
| | GAA | G | (SEQ ID | AAGAGA |
| | (SEQ ID | (SEQ ID | NO. 3) | GAGTTT |
| | NO. 40) | NO. 24) | | TTTTTT |
| | | | | T |
| | | | | (SEQ ID |
| | | | | NO. 33) |
| 3′-5′G | CCCG | AAGA | TTTTT | CCCGGT |
| Rev 9 | GTAGA | GAGA | TTTTT | AGAAAA |
| | A | G | (SEQ ID | GAGAGA |
| | (SEQ ID | (SEQ ID | NO. 3) | GTTTTT |
| | NO. 41) | NO. 24) | | TTTTT |
| | | | | (SEQ ID |
| | | | | NO. 34) |
| 3′-5′G | CCGGT | AAGA | TTTTT | CCGGTA |
| Rev 10 | AGAA | GAGA | TTTTT | GAAAAG |
| | (SEQ ID | G | (SEQ ID | AGAGAG |
| | NO. 42) | (SEQ ID | NO. 3) | TTTTTT |
| | | NO. 24) | | TTTT |
| | | | | (SEQ ID |
| | | | | NO. 35) |
| 3′-5′G | CACAC | AAGA | TTTTT | CACACG |
| Rev 11 | GGTA | GAGA | TTTTT | GTAGAA |
| | GAA | G | (SEQ ID | AAGAGA |
| | (SEQ ID | (SEQ ID | NO. 3) | GAGTTT |
| | NO. 43) | NO. 24) | | TTTTTTT |
| | | | | (SEQ ID |
| | | | | NO.36) |
| 3′-5′G | CCCC | AAGA | TTTTT | CCCCCC |
| Rev 12 | CCGG | GAGA | TTTTT | GGTAAG |
| | T | G | (SEQ ID | AGAGAG |
| | (SEQ ID | (SEQ ID | NO. 3) | TTTTTT |
| | NO. 44) | NO. 24) | | TTTT |
| | | | | (SEQ ID |
| | | | | NO. 37) |
| 3-5′G | CCCC | AAGA | TTTTT | CCCCCG |
| Rev 13 | CGGT | GAGA | TTTTT | GTAAGA |
| | (SEQ ID | G | (SEQ ID | GAGAGT |
| | N0.45) | (SEQ ID | NO. 3) | TTTTTT |
| | | NO. 24) | | TTT |
| | | | | (SEQ ID |
| | | | | NO. 38) |
| 3′-5′G | CCCC | AAGA | TTTTT | CCCCGGT |
| Rev 14 | GGT | GAGA | TTTTT | AAGAGAG |
| | (SEQ ID | G | (SEQ ID | AGTTTTT |
| | NO. 46) | (SEQ ID | NO. 3) | TTTTT |
| | | NO. 24) | | (SEQ ID |
| | | | | NO. 39) |

The control probes (3′5′ G Rev1 to Rev14) having a 3′-end poly(T)10 linker sequence were analyzed for self-dimers and hairpins and tested for interacting with AraH1 SPN (SEQ ID NO.1) and the effects on the interaction of AraH1 SPN with its target allergen AraH1. The results are shown in Table 6.

TABLE 6
Characterization of control probes
with 3′-linker for UV cross-linking

| | Self-dimer | Hairpin | To AraH1 - 1st | To AraH1 - 2nd |
|---|---|---|---|---|
| Probe | (Kcal/mol, | Tm | (Kcal/mol, | (Kcal/mol, |
| name | 50 mM Na) | (° C.) | 50 mM Na) | 50 mM Na) |
| 3′-5′G Rev1 | −17.03 | 42.6 | −27.4 | −15.35 |
| 3′-5′G Rev2 | −9.75 | 21.3 | −26.44 | −15.35 |
| 3′-5′G Rev3 | −9.75 | 37.2 | −26.44 | −15.35 |
| 3′-5′G Rev4 | −9.75 | 21.6 | −26.44 | −18.42 |
| 3′-5′G Rev5 | −9.75 | 30.8 | −27.4 | −15.35 |
| 3′-5′G Rev6 | −9.75 | 21.3 | −27.4 | −15.35 |
| 3′-5′G Rev7 | −9.75 | 21.3 | −27.4 | −15.35 |
| 3′-5′G Rev8 | −9.75 | 20.1 | −20.24 | −9.43 |
| 3′-5′G Rev9 | −9.75 | 20.1 | −20.24 | −6.68 |
| 3′-5′G Rev10 | −9.75 | 20.1 | −17.17 | −6.68 |
| 3′-5′G Rev11 | −5.83 | 20.1 | −14.1 | −13.27 |
| 3′-5′G Rev12 | −9.75 | 9.9 | −24.33 | −15.35 |
| 3′-5′G Rev13 | −9.75 | −2.7 | −21.26 | −12.28 |
| 3′-5′G Rev14 | −9.75 | −14.9 | −18.19 | −9.21 |

Another set of control probes with a poly(T)10 linker sequence tagged to the 5′ end of the oligonucleotide was designed. A spacer sequence (e.g., selected from Table 7) was inserted between the control oligonucleotide and the linker sequence.

TABLE 7
Control probe sequences with a
5′-end linker sequence

| | | | 5′G | Optimized |
|---|---|---|---|---|
| Control | | | control | probe |
| probe | Linker | Spacer | probe | sequence |
| revision | (5′-3′) | sequence | (5′-3′) | (5′-3′) |
| 5′-5′G | T | AAAAA | CCCCC | CCCCC |
| Rev 1 | (SEQ ID | (SEQ ID | CCGGT | CGGT |
| | NO. 3) | NO. 9) | (SEQ ID | (SEQ ID |
| | | | NO. 22) | NO. 47) |
| 5′-5′G | TTTTT | / | CCCCC | GGT |
| Rev2 | TTTTT | | CCGGT | (SEQ ID |
| | (SEQ ID | | (SEQ ID | NO. 48) |
| | NO. 3) | | NO. 22) | |
| 5′-5′G | TTTTT | CCAAC | CCCCC | TTTTT |
| Rev3 | TTTTT | CAACC | CCGGT | TTTTT |
| | (SEQ ID | (SEQ ID | (SEQ ID | CCAAC |
| | NO. 3) | NO. 7) | NO. 22) | CAACC |
| | | | | CCCCC |
| | | | | CCGGT |
| | | | | (SEQ ID |
| | | | | NO. 49) |
| 5′-5′G | T(SEQ ID | CCCCC | CCCCC | CCCCC |
| Rev4 | NO. 3) | CCCCC | CCGGT | CCCCC |
| | | (SEQ ID | (SEQ ID | GGT |
| | | NO. 5) | NO. 22) | (SEQ ID |
| | | | | NO. 50) |
| 5′-5′G | TTTTT | GGAAG | CCCCC | TTTTT |
| Rev5 | TTTTT | GAAA | CCGGT | TTTTT |
| | (SEQ ID | (SEQ ID | (SEQ ID | GGAAG |
| | NO. 3) | NO. 10) | NO. 22) | GAAAC |
| | | | | CCCCC |
| | | | | CGGT |
| | | | | (SEQ ID |
| | | | | NO. 51) |
| 5′-5′G | T | GAGAG | CCCCC | GAACC |
| Rev6 | (SEQ ID | AGAA | CCGGT | CCCCC |
| | NO. 3) | (SEQ ID | (SEQ ID | GGT |
| | | NO. 11) | NO. 22) | (SEQ ID |
| | | | | NO. 52) |
| 5′-5′G | T | GAGAG | CCCCC | GAAAC |
| Rev7 | (SEQ ID | AGAAA | CCGGT | CCCCC |
| | NO. 3) | (SEQ ID | (SEQ ID | CGGT |
| | | NO. 12) | NO. 22) | (SEQ ID |
| | | | | NO. 53) |

These 5′-5′G control probes were tested for intramolecular self-dimer and hairpin formations. Table 8 lists the testing results.

TABLE 8
Characterization of control probes
with 5′ linker for UV cross-linking

| | Self-dimer | Hairpin Tm |
|---|---|---|
| Probe names | (Kcal/mol, 50 mM Na) | (° C.) |
| 5′-5′G Rev1 | −16.52 | 35.9 |
| 5′-5′G Rev2 | / | / |
| 5′-5′G Rev3 | / | / |
| 5′-5′G Rev4 | / | / |
| 5′-5′G Rev5 | −9.75 | 38.7 |
| 5′-5′G Rev6 | −9.75 | 38.7 |
| 5′-5′G Rev7 | −9.75 | 38.7 |

The probes (AraH1 SPN probes and control probes) were diluted in a spotting buffer at a concentration of 25 uM. Following spotting the solutions to a COP plastic chip, the chip was exposed to UV light. After washing, the resulted nucleic acid chips were tested for hybridization signal. The targeting SPN (SEQ ID NO. 1) with a fluorescent marker was added for signal detection. The probe including the classic poly(C)(10) poly(A)(5) spacer sequence (i.e., SEQ ID NO. 13) resulted in a high level of cross reactivity with the control region of the target nucleic acid sequence, i.e., the SPN of SEQ ID NO. 1. The fluorescence signal is weak indicating a low UV grafting efficiency of the probe (AraH1 SPN probe version 1).

These different revisions of detection probes (Table 2) and control probes (Tables 4, 5 and 7) were screened for their assay performance. The data support that the revision 8 of the nucleic acid probe (SEQ ID NO. 20), and the revisions 6 and 13 of the control probe (SEQ ID NO. 31 and SEQ ID NO. 38) increased UV grafting efficiency and resulted in a reduced cross-reactivity with the control region of the SPN (SEQ ID NO. 1). These modified probes decrease self-dimer and hairpin stability as well, thereby increasing the immobilization efficiency.

The data indicates that the spacer sequences 5′GAGAGAGAA3′ (SEQ ID NO. 11) and 5′AAGAGAGAG3′ (SEQ ID NO. 24) can decrease the tendency of forming intramolecular self-dimer and hairpin structures, keeping the oligonucleotide as linear, thereby increasing immobilization efficiency.

### Example 2: Optimized Nucleic Acid Probes Specific to Control Sequence: PC60

Revisions of nucleic acid probes specific to a peanut control polynucleotide (PC60) (5′TAGGGAAGAGAAGGACATATGATCGTACCGCAAGTGACGTGTCCGTGCCGTG ATTGACTAGTACATGACCACTTGA3′; SEQ ID NO. 54) were tested for UV cross-linking. This control sequence (PC60) binds to peanut control materials, but not to peanut. A uniquely specific oligonucleotide probe sequence (5′TCAAGTGGTCAT3′; SEQ ID NO. 55) that is complementary to the sequence of PC60 (SEQ ID NO. 54, positions 65 to 77) was modified to have a poly(T) linker sequence (SEQ ID NO. 3) and a spacer sequence (e.g., selected from Table 9) at the 5′ end of the probe sequence.

TABLE 9
Nucleic acid probes specific to PC60

| | | | Probe | |
|---|---|---|---|---|
| | Linker | Spacer | sequence | Sequence |
| Probe | (5′-3′) | (5′-3′) | (5′-3′) | (5′-3′) |
| PC_3_1 | TTTTT | CCCCCC | TCAAGTG | TTTTTTT |
| _2 Rev 1 | TTTTT | CCCCAA | GTCAT | TTTCCCC |
| | (SEQ ID | AAA | (SEQ ID | CCCCCCA |
| | NO. 3) | (SEQ ID | NO. 55) | AAAATCA |
| | | NO. 4) | | AGTGGTC |
| | | | | AT |
| | | | | (SEQ ID |
| | | | | NO. 58) |
| PC_3_1 | TTTTT | CCCCC | TCAAGTG | TTTTTTT |
| _2 Rev2 | TTTTT | CCCC | GTCAT | TTTCCCC |
| | (SEQ ID | C | (SEQ ID | CCCCCCT |
| | NO. 3) | (SEQ ID | NO. 55) | CAAGTGG |
| | | NO. 5) | | TCAT |
| | | | | (SEQ ID |
| | | | | NO. 59) |
| PC_3_1 | TTTTT | CAAA | TCAAGTG | GTGGTCA |
| _2 Rev3 | TTTTT | (SEQ | GTCAT | T |
| | (SEQ ID | ID NO. | (SEQ ID | (SEQ ID |
| | NO. 3) | 56) | NO. 55) | NO. 60) |
| PC_3_1 | TTTTT | CAAAAC | TCAAGTG | AAGTGG |
| 1 Rev4 | TTTTT | (SEQ | GTCAT | TCAT |
| | (SEQ ID | ID NO. | (SEQ ID | (SEQ ID |
| | NO. 3) | 57) | NO. 55) | NO. 61) |
| PC_3_1 | TTTTT | CC | TCAAGTG | TTTTTT |
| _2 Rev 5 | TTTTT | (SEQ ID | GTCAT | TTTTCC |
| | (SEQ ID | NO. 8) | (SEQ ID | TCAAGT |
| | NO. 3) | | NO. 55) | GGTCAT |
| | | | | (SEQ ID |
| | | | | NO. 62) |
| PC_3_1 | TTTTT | AAAAA | TCAAGTG | TTTTTTT |
| _2 Rev6 | TTTTT | (SEQ | GTCAT | TTTAAAA |
| | (SEQ ID | ID | (SEQ ID | ATCAAGT |
| | NO. 3) | NO. 9) | NO. 55) | GGTCAT |
| | | | | (SEQ ID |
| | | | | NO. 63) |

These probe candidates are analyzed for self-dimers and hairpins and tested for interacting with PC60 (SEQ ID NO. 54). The probes are diluted in a spotting buffer. Following spotting the solutions to a COP plastic chip, the chip is exposed to UV light. After washing, the resulted nucleic acid chips are tested for hybridization signal. The targeting SPN (SEQ ID NO. 54) with a fluorescent marker was added for signal detection.

### Example 3: Fiducial Sequences

A group of fiducial oligonucleotide sequences (Table 10) are tested for UV cross-linking efficiency on a plastic chip and signal background. The fiducial sequence with the least background will be immobilized to a chipannel, forming a fiducial panel to normalize background noise in a detection assay.

TABLE 10
Fiducial sequences

| | Sequences with |
|---|---|
| Fiducial probes | tags (5′-3′) |
| Amine Cy5 | /5AmMC12/GAAAAGT |
| Fiducial_Revl | GCTCTGTGAACTCTA |
| | T/3Cv5Sp/ |
| | (SEQ ID NO. 64) |
| TC_TAGCy5 | TTTTTTTTTTGAAAAG |
| Fiducial_Revl | TGCTCTGTGAACTCTA |
| | T/3Cy5Sp/ |
| | (SEQ ID NO. 65) |
| TCTAGCy5 | TTTTTTTTTTAAAA |
| Fiducial_Rev2 | A/3Cy5Sp/ |
| | (SEQ ID NO. 66) |
| TC TAG_Rev1 | TTTTTTTTTTAAAAA |
| | (SEQ ID NO. 67) |
| TC_T AG_ | TTTTTTTTTTGAAAA |
| Spacer_Rev2 | GTGCTCTGTGAACTC |
| | TAT |
| | (SEQ ID NO. 68) |

### Example 4: DNA Chipannels for Food Test

Spotting buffers and wash buffers were optimized to minimize spots rolling caused by higher occurrence of surface defect and to improve UV grafting efficiency of DNA probes attached to a plastic chip (e.g., chipannel). The probe candidates were diluted in the spotting buffer at a concentration ranging from 0.1 μM to 40 μM, and immobilized on injection molded COP plastic to make a chipannel.

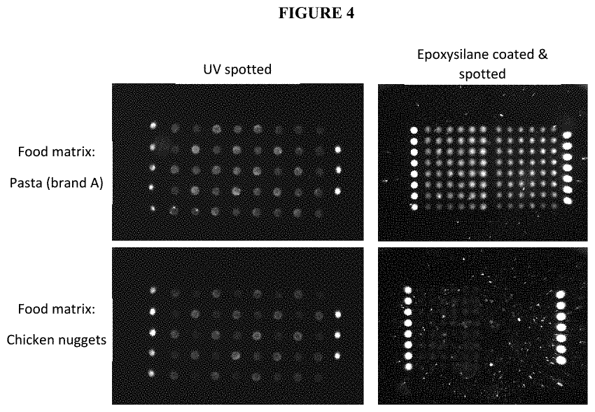

A food test was performed to test the UV printed DNA COP chipannels. After incubation with processed food samples, the chipannels were washed using an optimized wash buffer (II). The data indicate that the UV printed COP plastic chipannels showed very low adhesion to food residues (FIG. 4), while a DNA chipannel made by epoxysilane coated chip showed a much higher adhesion to food residues.

10 foods and foods spiked with 12.5 ppm peanut were tested using chipannels that were made by UV cross-linking of optimized nucleic acid probes and control probes, as discussed in Example 1, to a COP plastic chip to form a reaction panel and a control panel (e.g., as shown in FIG. 3). The results indicated that all foods displayed at least a 20% decrease in signal in the presence of peanut in the reaction panel that comprises a nucleic acid probe specific to AraH1 SPN (i.e., AraH1 SPN probe revision 8; SEQ ID NO. 20).

1. A nucleic acid chip comprising a solid substrate with at least one nucleic acid probe immobilized thereto, wherein the nucleic acid probe is composed of,
(a) a poly(T) linker sequence,
(b) a spacer sequence, and
(c) a uniquely specific oligonucleotide probe sequence that is complementary to the sequence or a portion of the sequence of a target nucleic acid molecule.

2. The nucleic acid chip of claim 1 wherein the solid substrate is a polymer chip.

3. The nucleic acid chip of claim 2 wherein the chip further comprises a control probe immobilized thereto.

4. The nucleic acid chip of claim 3 wherein the target nucleic acid sequence is an aptamer or derivative thereof, which comprises a sequence that specifically binds to an analyte of interest.

5. The nucleic acid chip of claim 4 wherein the probes are immobilized to the chip by UV light cross-linking.

6. The nucleic acid chip of claim 4 wherein the analyte of interest is a bacterium, a virus, a cell, a nucleic acid molecule, a protein, a lipid, a sugar and a compound.

7. The nucleic acid chip of claim 6 wherein the analyte is an allergen protein.

8. The nucleic acid chip of claim 1 wherein the poly(T) linker sequence comprises 5-20 T nucleotides.

9. The nucleic acid chip of claim 8 wherein the spacer sequence comprises about 5-15 nucleotides and wherein the spacer sequence does not affect the structural state of the uniquely specific oligonucleotide probe sequence.

10. The nucleic acid chip of claim 9 wherein the spacer comprises a sequence selected from the group consisting of SEQ ID Nos. 4-12, 23-25 and 56-57.

11. The nucleic acid chip of claim 3 wherein the nucleic acid probe comprises a sequence selected from the group consisting of SEQ ID NOs. 13-21, 58-63 and 70.

12. The nucleic acid chip of claim 11 wherein the control probe comprises a sequence selected from the group consisting of SEQ ID NOs. 26-39 and 47-53.

13. The nucleic acid chip of claim 3 wherein the chip further comprises a fiducial sequence.

14. An oligonucleotide comprising:
a poly(T) linker sequence;
a spacer sequence; and
a uniquely specific oligonucleotide probe sequence that is complementary to the sequence or a portion of the sequence of a target nucleic acid molecule.

15. The oligonucleotide of claim 14 wherein the target nucleic acid molecule comprises a nucleic acid sequence that binds specifically to an analyte of interest in a sample.

16. The oligonucleotide of claim 15 wherein the analyte of interest is a bacterium, a virus, a cell, a nucleic acid molecule, a protein, a lipid, a sugar and a compound.

17. The oligonucleotide of claim 16, wherein the analyte is an allergen protein.

18. The oligonucleotide of claim 14 wherein the poly(T) linker sequence comprises 5-20 T nucleotides.

19. The oligonucleotide of claim 18 wherein the linker sequence comprises a sequence presented by SEQ ID NO. 3.

20. The oligonucleotide of claim 14 wherein the spacer sequence comprises about 5-15 nucleotides and wherein the spacer sequence does not affect the structural state of the uniquely specific oligonucleotide probe sequence.

21. The oligonucleotide of claim 20 wherein the spacer comprises a sequence selected from the group consisting of SEQ ID Nos. 4-12, 23-25 and 56-57.

22. The oligonucleotide of claim 21 wherein the spacer sequence comprises a sequence presented by SEQ ID NO. 11.

23. An oligonucleotide probe for capturing a signaling polynucleotide that binds to an allergen comprising:
a linker sequence comprising a sequence presented by SEQ ID NO. 3;
a spacer sequence comprising a sequence selected from the group comprising SEQ ID Nos. 4-12; and
a uniquely specific oligonucleotide probe sequence that is complementary to the sequence or a portion of the sequence of the signaling polynucleotide, wherein the signaling polynucleotide comprises a sequence presented by SEQ ID NO.1.

24. The oligonucleotide probe of claim 23 wherein the uniquely specific oligonucleotide probe sequence comprises a sequence presented by SEQ ID NO. 2 or SEQ ID NO. 69.

25. The oligonucleotide probe of claim 24 wherein the probe comprise a sequence presented by SEQ ID NO. 20 or SEQ ID NO. 70.

26. A method for detecting an analyte of interest in a sample comprising,
(a) providing a complex formed from
(i) a sample suspected of containing the analyte of interest and (ii) a nucleic acid based detection agent in a condition allowing the binding of the analyte to the detection agent, wherein the detection agent comprises a nucleic acid sequence that binds to the analyte of interest;
(b) contacting the complex of the analyte of interest and the detection agent to a nucleic acid probe immobilized to a solid substrate, wherein the probe comprises an oligonucleotide probe sequence that is complementary to the sequence or a portion of the sequence of the detection agent;
(c) applying a detection module to the solid substrate for detecting a signal from the detection agent and the oligonucleotide probe, wherein if the analyte is not present in the sample, the detection agent not bound to the analyte is coupled to the solid substrate via the direct hybridization between the probe sequence and the target sequence of the detection agent; and
(d) measuring the amount of the detection agent wherein the amount of the detection agent indicates whether or not the analyte of interest is present in the sample.

27. The method of claim 26 wherein the nucleic acid probe further comprises a linker sequence and a space sequence.

28. The method of claim 27 wherein the solid substrate further comprises a control probe immobilized thereto.

29. The method of claim 28 wherein the nucleic acid probe and control probe are immobilized on the surface in a checkerboard pattern.

30. The method of claim 29 wherein the solid substrate further comprises a fiducial panel that is loaded a fiducial sequence.
